(19) United States
(12) Patent Application Publication
(10) Pub. No.: US 2007/0212678 A1
(43) Pub. Date: Sep. 13, 2007

(54) Method And Apparatus For Predicting Aggregation Kinetics Of A Biologically Active Material

(75) Inventors: Richard L. Remmele Jr, Carmarillo, CA (US); David J. Balaban, Camarillo, CA (US); Jian Zhang, Simi Valley, CA (US); Alexander Shoshitaishvili, Thousand Oaks, CA (US); Mark J. Durst, San Leandro, CA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AMGEN INC., Thousand Oaks, CA (US)

(21) Appl. No.: 11/587,168

(22) PCT Filed: Apr. 22, 2005
(86) PCT No.: PCT/US05/13928

§ 371 Date: Oct. 23, 2006

Related U.S. Application Data
(60) Provisional application No. 60/564,878, filed on Apr. 23, 2004.

(51) Int. Cl. C12Q 1/00; G06F 19/00

(52) U.S. Cl. 435/4; 702/19

(57) ABSTRACT

A mechanistic model was developed to extract meaningful thermodynamic and kinetic parameters from an irreversibly denatured process. As a result, methods and computer apparatus have been created that can be used to mathematically determine parameters that are predictive of aggregation kinetics of biologically active materials. Those parameters can then be used to predict stability or aggregation kinetics as a function of time and temperature.

### FIELD OF THE INVENTION

The present invention relates to methods and computer products for simulating and predicting properties of compositions comprising biologically active materials. In particular, the invention relates to methods for rigorously determining kinetic and thermodynamic parameters for multi-state processes as well as methods for using such parameters to determine, for example, stability and shelf-life.

### BACKGROUND OF THE INVENTION

Aggregation of proteins can occur as a consequence of conformational alterations attributed to denaturation. See, Joly, M. A Physico-Chemical Approach to the Denaturation of Proteins; Academic Press, Inc., London, 1965. In this context, the term “denaturation” refers to “a process (or sequence of processes) in which the conformation of polypeptide chains within the molecule are changed from that typical of the native protein to a more disordered arrangement”. Denaturation can result when a protein is conformationally perturbed by temperature, pH, or chemical denaturants. See, e.g., Remmele, R. L., Jr.; Bhat, S. D.; Phan, D. H.; Gombotz, W. R. Biochemistry 1999, 38, 5241-5247; Kauzmann, W. Some factors in the interpretation of protein denaturation Adv. Protein. Chem. 1959, 14, 1-64; and Speed-Ricci, M.; Sarkar, C. A.; Fallon, E. M.; Lauffenburger, D. A.; Brems, D. N. Protein Science 2003 12, 1030-1038.

Aside from compromising the integrity of the protein, aggregation can often lead to decreased solubility and elicit immunogenic responses in therapeutic settings. See, Pinckard, R. N.; Weir, D. M.; McBride, W. H. Clin. Exp Immunol., 1967, 2, 331-341; Moore, W. V.; Leppert, P. J. Clin. Endocrinology Metabolism, 1980, 51, 691-697; and Robbins, D. C.; Cooper, S. M.; Fineberg, S. E.; Mead, P. M. Diabetes, 1987, 36, 838-841. The importance in understanding this phenomenon, therefore, has broad implications not only in the realm of biochemistry, but also in the world of protein therapeutics.

Low confidence in estimates of the equilibrium parameters that control thermally irreversible systems has made it difficult to obtain thermodynamically meaningful information about such systems. Although there have been attempts to simplify the reactions, some of the approximations have depended upon the assumption that k₃>>k₂>>k₁. This assumption is only true for completely irreversible systems.

### SUMMARY

A more rigorous procedure has been developed for estimating the parameters that have relevance for reversible, partially reversible, and completely irreversible processes of thermal denaturing involving proteins. The derived parameters may apply not only to the thermodynamic aspects of the reactions, but also the kinetic aspects.

The process of protein aggregation can be characterized by thermodynamic and kinetic parameters. See, Krishnamurthy, R.; Manning, M. C. Current Pharmaceutical Biotechnology, 2002, 3, 361-371; and Grinberg, V. Y.; Burova, T. V.; Haertle, T.; Tolstoguzov, V. B. J. Biotechnology, 2000, 79, 269-280. The thermodynamic component characterizes the tendency for a given protein to unfold resulting in a change of state. In many cases, the unfolded state can lead to an irreversibly denatured aggregate state that is kinetically controlled. See, Vermeer, A. W. P.; Norde, W. Biophys. J., 2000, 78, 394-404 and Sánchez-Riuz, J. M.; López-Lacomba, J. L.; Cortijo, M.; Mateo, P. L. Biochemistry 1988, 27, 1648-1652. The kinetic component expresses how unfolding contributes to the overall mechanism leading to aggregation (or irreversibly denatured state).

In theory, aggregation is expected to be a second order process and therefore highly dependent on protein concentration. Moreover, because aggregation can involve multiple interactions between two or more molecules of protein, the aggregation reaction could in some cases be even greater than second order. Aggregation can also be rate limited by the formation of an aggregation-competent state that follows first order reaction kinetics.

The premise for unfolding-mediated aggregation can be explained with the knowledge that protein unfolding typically exposes buried hydrophobic regions of the molecule that become reactive in regard to associations between neighboring molecules that have also unfolded in like manner (via hydrophobic-hydrophobic interactions). See, Brandts, J. F. Thermobiology; Academic Press, 1967, Chapter 3, pp. 25-75. In this respect, the change in state is the transition from the compact or native state to the unfolded or conformationally denatured state. The kinetics that describe the system can indicate how susceptible the unfolded state is to the interaction between adjacent molecules to form complexes. Such systems were recognized by Lumry and Eyring and modeled as three-state systems that involved first an unfolding event followed by an irreversibly denaturing process. See, Lumry, R.; Eyring, H. J. Phys. Chem., 1954, 58, 110-120. The model may be described by the scheme below, 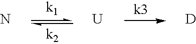
where N is the native state, U is the unfolded (conformationally denatured) state and D is the irreversibly denatured state or aggregation product of the reaction. The scheme represented can be described in terms of the kinetics associated with the rates of the forward and reverse reactions (k₁ and k₂) and for the irreversibly denatured or aggregate state (k₃). The kinetics of the equilibrium between the native and unfolded states are also related to the thermodynamics of the reaction since the equilibrium constant may be described as a function of the rates, k₁ and k₂,
K₁₂=[U]/[N]=k₁/k₂ (1)

Building upon this premise, it would seem that reactions that are dependent upon an unfolding step should exhibit non-Arrhenius profiles producing curvature in the vicinity of the denaturation temperature (or melting temperature, Tₘ).

The temperature of denaturation can be determined accurately using microcalorimetry when the system is fully reversible and when the scan rate does not exceed the rate of unfolding. In cases where the system is irreversible, the determination is more complicated and does not lend itself to thermodynamic treatment. Furthermore, if the kinetics of the aggregation process are dependent upon unfolding, a change in kinetic behavior that coincides with the Tₘ of the unfolding transition should be apparent. Finally, a microcalorimetric scan rate dependence of the Tₘ is expected if the scheme above is applicable and kinetically controlled.

One of the central limitations of classic or extended Lumry-Eyring theory for modeling protein aggregation rates, is the need to generalize reaction mechanisms. Give this limitation, is it possible to extract meaningful information about the important contributing parameters that govern protein aggregation rates? The idea that scan-rate dependent unfolding studies using microcalorimetry could be used to extract kinetic information from reactions that depend upon conformationally altered states had been proposed (but not experimentally tested) in the 1980's by Privalov and Potekhin. Approaches for extracting meaningful enthalpies from irreversible microcalorimetric experiments of protein unfolding has been previously reported. Sanchez-Ruiz and coworkers made the generalization that in a kinetically controlled process where the intermediate or “U” state was negligibly populated (as in the case for completely irreversible protein unfolding reactions where k₃>>k₁), the three-state Lumry-Eyring model could be simplified to approximate a first-order reaction from which reliable activation energies that followed Arrhenius behavior could be obtained. Later building upon this work, Lepock and coworkers applied a classic three-state Lumry-Eyring model to simulate varied rate constant perturbations imposed upon thermodynamic and kinetically controlled steps associated with the unfolding thermogram properties of microcalorimetry data. Finally, Christopher Roberts applied a more general theoretical approach taking into account 1^{st}, 2^{nd}, and higher order reaction kinetics ascribed to complex thermodynamic and kinetic properties of irreversible aggregation reactions in order to predict shelf-life. However, in his description, Roberts had not derived an expression for Cₚ (excess heat capacity) as a function of temperature and scan rate.

In contrast to these approaches, the present work derives a theoretical treatment obtained from simulating of scan-rate dependent microcalorimetry data in order to extract meaningful thermodynamic and kinetic parameters from a system that is predominantly irreversible and exhibits non-Arrhenius aggregation kinetics. This investigation elucidates the role of thermal unfolding as it pertains to an irreversibly aggregated process involving rhuIL-1R (II). The study of rhuIL-1R considers the case where aggregate formation results from the association of unfolded protein forms and describes protein unfolding as prerequisite through which dimers form, becoming the precursor to all higher order oligomerized states.

The present invention provides methods and computer products that might be used to predict stability of a biologically active material. In certain embodiments, the stability of the biologically active material depends, at least in part, upon a change in the conformational state of the material.

According to one embodiment, a composition comprising a biologically active material is provided. The biologically active material is capable of a conformational change due to a thermal change and is substantially in its native state prior to such thermal change. A conformational change of the biologically active material can be measured as a function of temperature and time. In certain embodiments, physical or chemical consequences of such change are measured. The measurements may be made under conditions that result in significant irreversible unfolding, for example in a predominantly irreversible, scan-rate dependent system.

Various thermodynamic and kinetic parameters can be determined for the biologically active material based on the measurement(s) of the conformational change and physical or chemical consequences of such change. These parameters may be predictive of aggregation kinetics. In certain embodiments, enthalpy or free energy of transition; ΔCₚ between native and denatured states of the material; and the temperature at which about 50% of the protein is in an unfolded state (and about 50% of the protein is in its native state) are determined. The parameters may collectively model non-Arrhenius aspects of aggregation kinetics. They may also model aggregation as a 2^{nd} order reaction involving two types of irreversibly unfolded states.

In certain embodiments of the invention, aggregation kinetics or the stability of the biologically active material can be predicted from these thermodynamic and kinetic parameters. For example, in some circumstances they may be used to predict aggregation kinetics as a function of time, temperature, and concentration. The parameters might also be used to determine different reaction rate constants or be extrapolated to determine shelf life of the biologically active material at one or more storage temperatures, or to establish a recommended storage temperature.

Methods and computer products are disclosed for predicting the effect of an excipient on shelf life of a biologically active material. One or more of the kinetic and thermodynamic parameters for the composition comprising the biologically active material can be compared to the analogous parameters for the composition comprising both one or more excipients of interest and the biologically active material to determine the effect of the excipient of interest on the shelf life of the biologically active material. Or, a property of a biologically active material that pertains to aggregation (such as rate or amount of chemical decomposition, proteolysis, hydrolysis, deamidation, or oxidation) might be predicted.

Briefly, the new approach includes a method for determining parameters for predicting aggregation kinetics of a biologically active material comprising the steps of:
(a) providing measurements of conformational change of the biologically active material at varying temperatures and varying times, and
(b) using the measurements of part (a) to mathematically determine activation energy parameters (E) and frequency factor parameters (A) associated with at least three different reaction rate constants, the parameters being predictive of aggregation kinetics of the biologically active material.

The biologically active material may be a hormone, cytokine, hematopoietic factor, growth factor, antibody, antiobesity factor, trophic factor, anti-inflammatory factor, antibody or enzyme, or erythropoietin, granulocyte-colony stimulating factor, stem cell factor, or leptin. It may be in a formulation that further comprises one or more excipients, and steps (a) and (b) may be carried out on a plurality of different formulations of the material, at least two of the formulations are at different pH.

The measuring can be carried out using differential scanning calorimetry or size exclusion chromatography. Conformational change of the biologically active material can be measured as a function of temperature varied uniformly over time.

Some of the parameters that are determined may collectively model non-Arrhenius aspects of the aggregation kinetics. The activation energy parameters (E) and frequency factor parameters (A) may be associated with at least four reaction rate constants, or with no more than four reaction rate constants.

Step (b) can include providing estimated activation energy and frequency factor parameters, calculating predicted measurements of conformational change based on the estimated parameters, and using an estimation method to compare predicted measurements to measurements from step (a), for example with a non-linear least squares fitting method. The approach may include applying a weighting factor dependent on the scan rate. Step (b) can also include may include determining enthalpy or free energy of transition, determining ΔCp, ΔCₚ^{D}^{₁}, and ΔCₚ^{D}^{₂} (that change in heat capacity being predictive of aggregation kinetics of the biologically active material); or determining the temperature at which about 50% of the protein is in an unfolded state and about 50% of the protein is in its native state. The approach can also involve modeling aggregation, as a function of time at different temperatures, as a first and second order reaction. It can also include evaluating identifiability and variability of one or more of the parameters.

One or more of the following equations is used:
\dot{N}=−k₁N+k₂U
\dot{U}=k₁N−(k₂+k₃)U−k₄U²
\dot{D}=k₃U+k₄U²

$$\begin{matrix}{C}_{P} ( v , T ) = ( Δ {H}_{m} + Δ {C}_{P} ( T - {T}_{m} ) ) ( - \frac{1}{v} N ) + Δ {C}_{P} U + \\ \frac{{k}_{3}}{v} ( {E}_{3} + Δ {C}_{P}^{{D}_{1}} ( T - {T}_{m} ) ) U + \frac{{k}_{4}}{v} ( {E}_{4} + {C}_{P}^{{D}_{2}} ( T - {T}_{m} ) ) {U}^{2} + \\ ( Δ {C}_{P}^{{D}_{1}} + Δ {C}_{P} ) {D}_{1} + ( Δ {C}_{P}^{{D}_{2}} + Δ {C}_{P} ) {D}_{2}\end{matrix}$$

A_{gg}(T,t)=D.

Once derived, the parameters can be used to predict stability or aggregation kinetics as a function of time and temperature using at least three different reaction rate constants. For example, the level of aggregation of the biologically active material as a function of temperature, time, and concentration of said biologically active material might be determined, or might be determined at a given temperature, for example at a temperature of 40 degrees C. or less, or in a range from 4 to 25 degrees C., or from 15 to 30 degrees C., or from −5 to 15 degrees C., or from 2 to 8 degrees C. The level of aggregation might be predicted after a time period, for example a time of six months or more, or one year or more, or two years or more. Or, it may involve predicting time to reach an unacceptable level of aggregation, such as the time to reach 50% aggregation. It might also involve predicting aggregation half-life of the biologically active material (with or without incipients) as a function of temperature and concentration of the biologically active material, predicting shelf-life of the material at one or more storage temperatures, or predicting an optimal storage temperature. It might also be used to predict stability or level of aggregation for a plurality of formulations of the material, or for formulations that contain one or more excipients.

### BRIEF DESCRIPTION OF THE DRAWINGS

FIG. 1 is an illustration of unnormalized SEC results of rhuIL-1R(II) at 58° C. showing the progression of aggregates over time in seconds. Additionally, the arrows indicate the changes in the monomer (downward) and the aggregate (upward). The vertical lines bracketing elution times between 5.7 minutes and 8.4 minutes represent the integration region describing total aggregation. The eluting component near 8 minutes is assigned to the dimer population. It is relatively stable at different temperatures and time, thus supporting the pseudo-steady-state aggregation mechanism.

FIG. 2 is a pair of proposed enthalpy and free energy diagrams describing the thermal unfolding and aggregation of rhuIL1R(II). N represents the native state; U, the unfolded state; D1, aggregates formed through 1^{st} order reactions; and D2, aggregates from 2^{nd} order reactions. D₁* and D₂* are the corresponding transition states leading to aggregation. Activation free energies related to each state are denoted as ΔGᵢ^{‡} (where i=1, 2, 3, or 4.) The free energy of unfolding is ΔG₁₂ and aggregation free energy is ΔG_{agg}. As temperature approaches Tm, ΔG₁₂ goes to zero.

FIG. 3 is a comparison of the calculated result using eqn (31) (solid lines) with experimental data (broken lines) for the DSC scan rate dependent experiment. From left to right, the scanning rates are 0.25, 0.5, 1.0, and 1.5° C./min, respectively. The inset figure is a DSC scan for a 91% reversible rhuIL-1R(II) system in 0.1 M sodium phosphate plus 2 M urea at pH=7.0. In all data shown the transition baseline has been subtracted.

FIG. 4 is a pair of charts of the time-temperature data of aggregation. The predicted fits based upon the model (eqn (20)) are depicted by the solid lines. Data points represented by the assorted symbols are the experimentally determined values. In the top figure, the temperatures from top to bottom are 75° C., 69° C., 65° C., 58° C., and 50° C. In the bottom figure, the temperatures from top to bottom are 39° C., 37° C., and 34° C.

FIG. 5 is a set of van't Hoff plots of data for rhuIL1R(II). The top plot (A) shows the best least squares fit of data to two distinctly different lines showing least squares equation for pre and post unfolding transition temperature zones. The cross over point of the two lines is in the vicinity of the Tₘ. The bottom plot (B) compares the initial aggregation rates with * being the calculation based on the model (eqn (21)); the dashed line being the rate approximation

$$( {k}^{′} = \frac{{k}_{1} {k}_{3}}{{k}_{1} + {k}_{2} + {k}_{3}} ) ;$$

and ∘ being the experimental data. The experimental error in temperature is indicated by ‘+’ on either side of the data points. The data denoted by ● is the Sánchez-Ruiz et al. treatment of the scan rate data.

FIG. 6 is a plot of the temperature dependence of the kinetic rate constants for 2 mg/mL rhuIL-1R(II). The point denoting the temperature where k₁=k₂ is the extracted Tₘ.

FIG. 7 is a comparison of the concentration dependence predicted by the model using the parameters extracted. The curves depict the behavior predicted by the model and the data points represent measured aggregation by SEC. The upper curve and points correspond to data at 67.5° C. for a duration of 2 minutes. The lower ones correspond to data at 42.5° C. for 48 hours.

FIG. 8 is a flowchart of the method used in one embodiment of the invention.

FIG. 9 is a flowchart of a method of evaluating identifiability of parameters obtained using the model.

FIG. 10 is a flow chart of a method of evaluating variability of parameters obtained using the model

FIG. 11 is a block diagram of a computer system that can be used to implement various aspects of this invention.

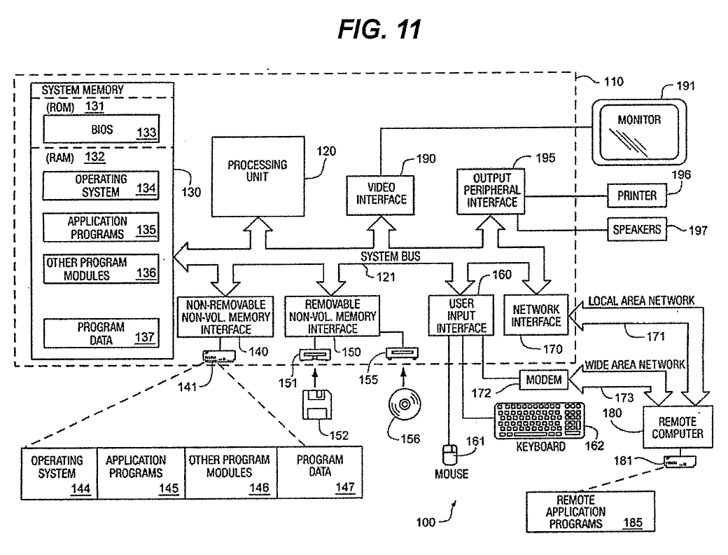

### DETAILED DESCRIPTION

Abbreviations and definitions will be provided before an overview and specific applications are discussed.

I. Abbreviations and Definition

The following abbreviations and definitions will be used.

A. Abbreviations

The following abbreviations are used:

T=temperature (in K unless otherwise noted)

t=time (in minutes unless otherwise noted)

G=Gibbs free energy (in kcal/mol)

S=entropy (in kcal/mol/K)

H=enthalpy (in kcal/mol)

h=enthalpy of the ensemble state (in kcal/mol)

E=activation energy

ε=energy of the ensemble state

K_{B}=the Bolzmann constant

R=the gas constant (0.0019872 kcal/mol/K)

DSC=differential scanning calorimetry

SEC=size exclusion chromatography

CD=circular dichroism

STP=standard temperature and pressure

Subscripts and superscripts are applied to indicate state.

This invention is not limited to particular methods, devices, or systems, which can, of course, vary. The terminology used herein is for the purpose of describing particular embodiments only, and is not intended to be limiting.

As used in this specification and the appended claims, the singular forms “a,” “an,” and “the” include plural referents unless the content clearly dictates otherwise. Thus, for example, reference to “a surface” includes a combination of two or more surfaces; reference to “protein” can include mixtures of protein, and the like.

Unless defined otherwise, all technical and scientific terms have the same meaning commonly understood by one of ordinary skill in the art to which the invention pertains.

B. Definitions

“Aggregation” refers to one of the most common protein degradation pathways which results in tangled and amorphous masses of protein fibers.

“Biologically active material” refers to a molecule that is capable of a conformational change and, in certain embodiments, unfolding, due to a thermal change.

“Denaturation” refers to a process in which the conformation of a molecule is changed from a first state to a more disordered arrangement. In a certain embodiment wherein the molecule is a protein, denaturation refers to a process (or sequence of processes) in which the conformation of polypeptide chains with the protein are changed from that typical of the native protein to a more disordered arrangement without cleavage of any of the primary chemical bonds that link one amino acid to another. Cleland et al. Critical Reviews in Therapeutic Drug Carrier Systems 10(4): 307-377 (1993).

“Denatured nucleic acid” refers to a nucleic acid that has been treated to remove folded, coiled, or twisted structure. Denaturation of a triple-stranded nucleic acid complex is complete when the third strand has been removed from the two complementary strands. Denaturation of a double-stranded DNA is complete when the base pairing between the two complementary strands has been interrupted and has resulted in single-stranded DNA molecules that have assumed a random form. Denaturation of single-stranded RNA is complete when intramolecular hydrogen bonds have been interrupted and the RNA has assumed a random, non-hydrogen bonded form.

“Denatured protein” refers to a protein which has been treated to remove secondary, tertiary, or quaternary structure.

“Differential scanning calorimetry” refers to an analytical method based upon the detection of changes in the heat content or the specific heat of a sample with temperature.

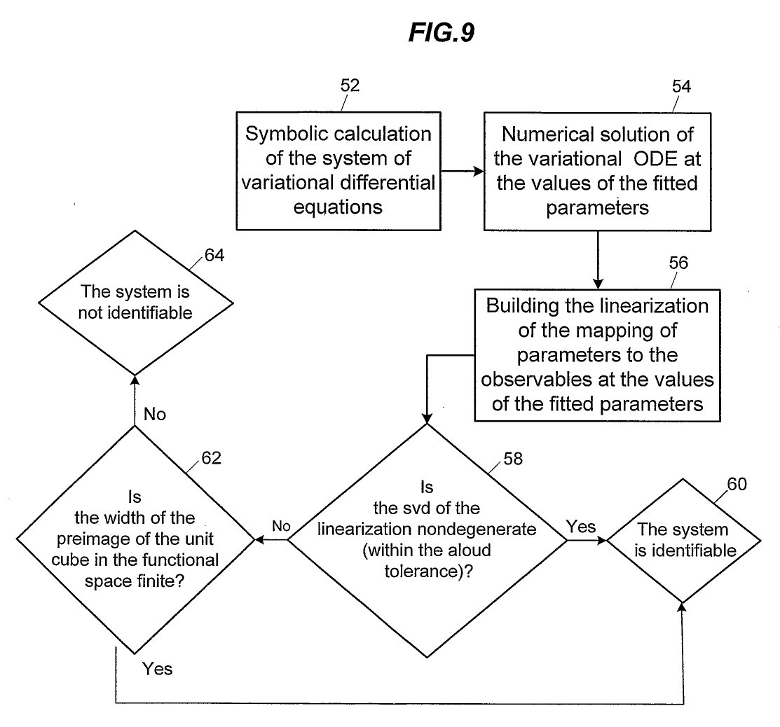

“Evaluating identifiability” of parameters refers to evaluating the uniqueness of the parameters. For example, a first set of parameters is not considered identifiable if a different second set of parameters can result in the same predicted measurements of conformational change that would be predicted by the first set of parameters. One embodiment of a method of evaluating identifiability of parameters is illustrated in FIG. 9.

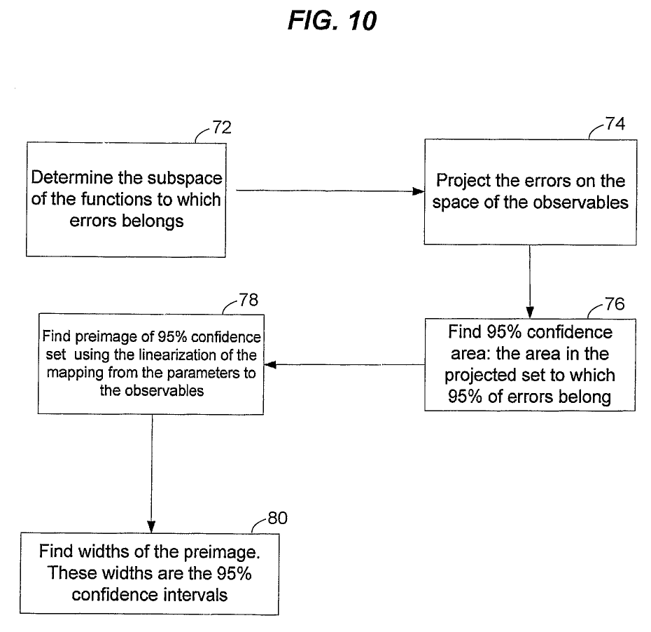

“Evaluating variability” of parameters refers to evaluating how variability in actual measurements affects the parameters determined from these measurements of conformational change. In such an evaluation, it is assumed that the parameters are identifiable. For example, adding random variability (i.e. “noise”) to the measurements should result in some but relatively little change in the determined parameters; from this, it is possible to determine a 95% confidence interval for a range of parameter values. One embodiment of a method of evaluating variability of parameters is illustrated in FIG. 10.

“Folding,” “refolding,” and “renaturing” refer to the acquisition of the correct secondary, tertiary, or quaternary structure, of a protein or a nucleic acid, which affords the full chemical and biological function of the biomolecule.

“Glass,” “glassy state,” or “glassy matrix,” refers to a liquid that has lost its ability to flow, i.e. it is a liquid with a very high viscosity, wherein the viscosity ranges from 10¹⁰ to 10¹⁴ pascal-seconds. It can be viewed as a metastable amorphous system in which the molecules have vibrational motion but have very slow (almost immeasurable) rotational and translational components. As a metastable system, it is stable for long periods of time when stored well below the glass transition temperature. Because glasses are not in a state of thermodynamic equilibrium, glasses stored at temperatures at or near the glass transition temperature relax to equilibrium and lose their high viscosity. The resultant rubbery or syrupy, flowing liquid is often chemically and structurally destabilized.

The “glass transition temperature” is represented by the symbol T_{g} and is the temperature at which a composition changes from a glassy or vitreous state to a syrup or rubbery state. Generally, T_{g} is determined using differential scanning calorimetry and is standardly taken as the temperature at which onset of the change of heat capacity (Cp) of the composition occurs upon scanning through the transition. There is no present international convention for the definition of T_{g}. The T_{g} can be defined as the onset, midpoint, or endpoint of the transition; for purposes of this invention, the midpoint of the transition will be used. See the article by C. A. Angell, Science, 267, 1924-1935 (1995) and Jan P. Wolanczyk, Ciyo-Letters, 10, 73-76 (1989). For detailed mathematical treatment see Gibbs and DiMarzio, Journal of Chemical Physics, 28, No. 3, 373-383 (March, 1958). These articles are incorporated herein by reference.

A “higher stability” is indicated by a lower aggregation rate or a longer shelf-life. An alternative exemplary measure of stability is the free energy (ΔG) of unfolding from a native state to a denatured state. A higher free energy indicates a more stable biologically active material. For example, a typical of ΔG for proteins is usually 5-25 kcal/mol, but a given ΔG for a particular protein can be increased by using a stabilizing formulation of biologically active material, e.g. by adding excipients, or adjusting pH or concentration of the biologically active material. “Lyophilize” or “lyophilization” or “freeze-dry” will refer to a process for the removal of water from frozen compositions by sublimation under reduced pressure.

“Measuring” a conformational change will include measuring the change directly or measuring the change indirectly by measuring a physical or chemical consequence of such change.

The “midpoint temperature” or “Tₘ” is the temperature midpoint of a thermal denaturation curve. The Tₘ can be readily determined using methods well known to those skilled in the art. See, for example, Weber, P. C. et al., J. Am. Chem. Soc. 116:2717-2724 (1994); Clegg, R. M. et al., Proc. Nati. Acad. Sci. U.S.A. 90:2994-2998 (1993).

“Native protein” refers to a protein which possesses the degree of secondary, tertiary, or quaternary structure that provides the protein with full chemical and biological function.

“Nucleic acid,” “oligonucleotide,” or grammatical equivalents herein mean a molecule comprising at least ten nucleotides covalently linked together. A nucleic acid will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochein. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. EngI. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research,” Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research,” Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176).

An “optimal formulation” is a formulation, including, for example, pharmaceutically acceptable excipients, pH and concentration of biologically active material, that exhibits higher stability relative to other formulations.

An “optimal storage temperature” is a recommended storage temperature range that provides higher stability relative to other typical storage temperatures. For example, refrigeration at 0-8 degrees C. may provide higher stability for the biologically active material relative to room temperature storage at 21-23 degrees C. “Pharmaceutically acceptable” excipients (vehicles, additives) are those which can reasonably be administered to a subject mammal to provide an effective dose of the active ingredient employed. Preferably, these are excipients that the Federal Drug Administration (FDA) has to date designated as Generally Regarded as Safe (GRAS).

“Pharmaceutical composition” refers to preparations that are in such a form as to permit the biological activity of the active ingredients to be unequivocally effective, and that contain no additional components that are toxic as administered to the subjects.

The terms “polypeptide,” “peptide,” and “protein” are used interchangeably herein to refer to a polymer of amino acid residues, and includes peptides, polypeptides, consensus molecules, antibodies, analogs, derivatives, or combinations thereof. The terms apply to amino acid polymers in which one or more amino acid residues is an artificial chemical analogue of a corresponding naturally occurring amino acid, as well as to naturally occurring amino acid polymers. Amino acids may be referred to herein by either their commonly known three letter symbols or by Nomenclature Commission. Nucleotides, likewise, may be referred to by their commonly accepted single-letter codes, i.e., the one-letter symbols recommended by the IUPAC-IUB. “Predicting stability” refers to the prediction of the ability of a composition comprising a biologically active material to retain its physical stability, chemical stability, and/or biological stability upon storage. Prediction encompasses the determination of a recommended storage temperature; the determination of shelf-life at one or more storage temperatures, including the recommended storage temperature; the determination of the amount or rate of aggregation of the biologically active material for temperatures and time periods of interest; and the determination of other parameters as will be appreciated by one of skill in the art.

“Predicting stability” refers to the prediction of the ability of a composition comprising a biologically active material to retain its physical stability, chemical stability, and/or biological stability upon storage. Prediction encompasses the determination of a recommended storage temperature; the determination of shelf-life at one or more storage temperatures, including the recommended storage temperature; the determination of the amount or rate of aggregation of the biologically active material for temperatures and time periods of interest; and the determination of other parameters as will be appreciated by one of skill in the art.

“Recommended storage temperature” for a composition is the temperature (T) at which a drug composition is to be stored to maintain the stability of the drug over the shelf life of the composition in order to ensure a consistently delivered dose. This temperature is initially determined by the manufacturer of the composition and approved by the governmental agency responsible for approval the composition for marketing (e.g., the Food and Drug Administration in the U.S.). This temperature will vary for each approved drug depending on the temperature sensitivity of the active drug and other materials in the product. The recommended storage temperature will vary from about −70° C. to about 40° C. but powdered and liquid drug compositions are generally recommended for storage between about 4° C. and about 25° C. Usually a drug will be kept at a temperature that is at or below the recommended storage temperature.

A biologically active material “retains its biological activity” in a pharmaceutical composition if the biological activity of the biologically active material at a given time can be within about 10% (within the errors of the assay) of the biological activity exhibited at the time the pharmaceutical composition was prepared as determined in a binding assay, for example.

A biologically active material “retains its chemical stability” in a pharmaceutical composition if the chemical stability at a given time is such that the biologically active material is considered to still retain its biological activity as defined above. Chemical stability can be assessed by detecting and quantifying chemically altered forms of the biologically active material. Chemical alteration may involve size modification (e.g. clipping of proteins) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by ion-exchange chromatography, for example.

A biologically active material “retains its physical stability” in a pharmaceutical composition if, e.g., aggregation, precipitation, and/or denaturation upon visual examination of color and/or clarity, or as measured by UV light scattering or by size exclusion chromatography, are not significantly changed.

“Size exclusion chromatography” refers to a separation technique in which separation mainly according to the hydrodynamic volume of the molecules or particles takes place in a porous non-adsorbing material with pores of approximately the same size as the effective dimensions in solution of the molecules to be separated

A “stable” formulation or composition is one in which the biologically active material therein essentially retains its physical stability, chemical stability, and/or biological activity upon storage. Various analytical techniques for measuring stability are available in the art and are reviewed, e.g., in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period.

“Shelf stability” or “shelf life” refers to the loss of specific activity and/or changes in secondary structure from the biologically active material over time incubated under specified conditions.

In a pharmacological sense, a “therapeutically effective amount” of a biologically active material refers to an amount effective in the prevention or treatment of a disorder wherein a “disorder” is any condition that would benefit from treatment with the biologically active material. This includes chronic and acute disorders or diseases including those pathological conditions which predispose a patient to the disorder in question.

A “thermal denaturation curve” is a plot of the physical change associated with the denaturation of a protein or a nucleic acid as a function of temperature. See, for example, Davidson et al, Nature Structure Biology 2:859 (1995); Clegg, R. M. et al., Proc. Natl. Acad. Sci. U.S.A. 90:2994-2998 (1993).

“Two-state process” refers to a process wherein the measured calorimetric enthalpy change is equivalent to the effective two-state van't Hoff enthalpy change. A “multi-state process” includes a two-state process and higher order processes.

II. Overview

The present invention provides methods for simulating multi-state processes of instability of proteins and other biologically active molecules. More specifically, when instability depends upon a change in conformational state of the molecule, the invention may enable one skilled in the art to rigorously determine kinetic and thermodynamic parameters related to that process. These parameters might then be used to predict an aggregation property (such as shelf-life) of the biologically active material, including for example, the effect of concentration, pH, or excipients on shelf-life.

This overview includes a brief description of materials and methods that were used in developing the invention, and is followed by a theoretical treatment.

A. Materials and Methods

The materials and methods that were used in developing the invention are described below.

1. Material

Purified rhuIL-1R(II) (recombinant human Interleukin-1 receptor, type II) was obtained as a bulk drug concentrate (˜10 mg/mL) in a phosphate buffered saline solution (PBS: 20 mM sodium phosphate (pH 7.4), 150 mM NaCl) obtained from Immunex Corporation (now Amgen, Inc.). The protein, expressed in CHO cells was approximately 20% glycosylated. Protein concentrations were determined spectrophotometrically at 280 nm using an experimentally determined molar extinction coefficient of 1.61 mL/mg-cm. All excipients used were reagent grade or better. The protein polypeptide molecular weight was approximately 38 kD.

2. Methods

Two studies were used in developing this invention, and are described more fully below.

a. Microcalorimetry (DSC Experiment)

Samples were evaluated in a vp-DSC (MicroCal, Inc.) using scan rates of 0.25, 0.5, 1.0 and 1.5° C./min. Protein solutions were fixed at 2 mg/mL (unless otherwise noted) by diluting with the PBS buffered solution. The Tₘ dependence on scan rate was assessed in the microcalorimeter using the method described by Sanchez-Ruiz and coworkers.

Thermal reversibility of a 2 mg/mL solution was also examined in PBS at a scan rate of 1° C./min within the time frame of differential heating to 90° C., followed by cooling, re-equilibrating, and subsequently reheating a second time (time lapse between scans was essentially 1 hr). The data were evaluated using Origin software (version 5.0) provided with the instrument.

b. Time-Temperature Aggregation Studies

The temperatures selected for the time-temperature aggregation studies covered a broad range that straddled the unfolding transition endotherm. The main idea was to traverse the transition region that included temperatures well outside the transition envelope above and below the apparent Tₘ (˜58° C. at a scan rate of about 1° C./min).

The kinetics of the aggregation reaction were studied by placing 0.5 mL of protein solution in a 2 mL capacity polypropylene eppendorf vial and heating the contents at a designated temperature in an appropriate heating device (either incubator or heating block) for a designated period of time. For studies conducted using a heating block, careful attention was given to temperature control (±1° C.) and uniform heating of the sample. Bored wells (1-cm inside diameter) in an aluminum block were filled with water and allowed to equilibrate at the desired temperature prior to insertion of the eppendorf vial. At elevated temperatures (>Tₘ) equilibrium was reached immediately. In the low temperature studies (<40° C.), heating experiments were carried out in the incubator and given adequate time (2 hrs) to reach the equilibrium temperature prior to starting the clock. This was established by direct monitoring of the sample when temperature exhibited no greater change than±1° C. at equilibrium with the surrounding environment. At designated time points samples were removed from the heating device, immediately placed on ice, and stored in the refrigerator before examination by size exclusion chromatography (SEC).

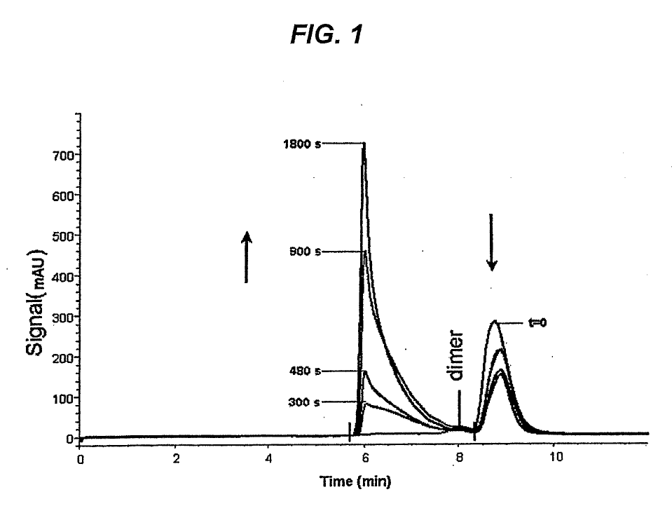

Analysis was carried out on a HP-1100 HPLC system. Samples were eluted off a TosoHaas TSK-G3000 SWXL column at 1 mL/min with 100 mM phosphate (pH 6.5), 50 mM NaCl eluent. A 20 μg sample injection load was used per HPLC run. The kinetics were determined by assessing the total amount of aggregation (expressed as a percentage of the total area under the sample protein peaks) at a designated time, as shown in FIG. 1 for the 58° C. data as an example. The region of integration was defined by the vertical lines bracketing the elution times extending from about 5.7 to 8.4 minutes (FIG. 1). All aggregation measurements were determined in the same way at other temperatures studied.

Attention to possible competing side reactions (i.e., breakdown) was investigated and found to be negligible (no evidence) throughout the time duration of the studies presented. Hence, one could be assured that the aggregation pathway was the primary instability being detected during the experiments. Furthermore, there was no evidence of protein insolubility in all cases studied herein.

Detection of the eluting components was achieved with a photodiode array detector monitoring absorbance at 220 nm. The main peak eluting near 9 minutes and the peak eluting at 8 minutes were confirmed to be monomer using the “three detector” light scattering method described previously. See, Wen, J.; Arakava, T.; Talvenheimo, J.; Weicher, A. A.; Horan, T.; Kita, Y.; Tseng, J.; Nicolson, M.; Philo, J. S. Techniques in Protein Chemistry 1996, VII, pp23-31 and Wen, J.; Arakava, T.; Philo, J. S. Analytical Biochemistry 1996, 240, 155-166. It should be noted that the SEC aggregation result is assumed to accurately reflect solution state composition.

B. Theoretical Treatment

The goal of the theoretical modeling in this work was to describe the dominant underlying physical processes on a macroscopic level, and to extract as accurately as possible the kinetic and thermodynamic parameters. Because many of the parameters are related to each other, it is important to incorporate these relationships in the model and to use some experimentally derived quantities to cross check the consistency.

In the next section, a description of the system is followed by discussions of the pertinent kinetic equations and reaction rates, and the observables that were used when developing the approach.

1. System Description

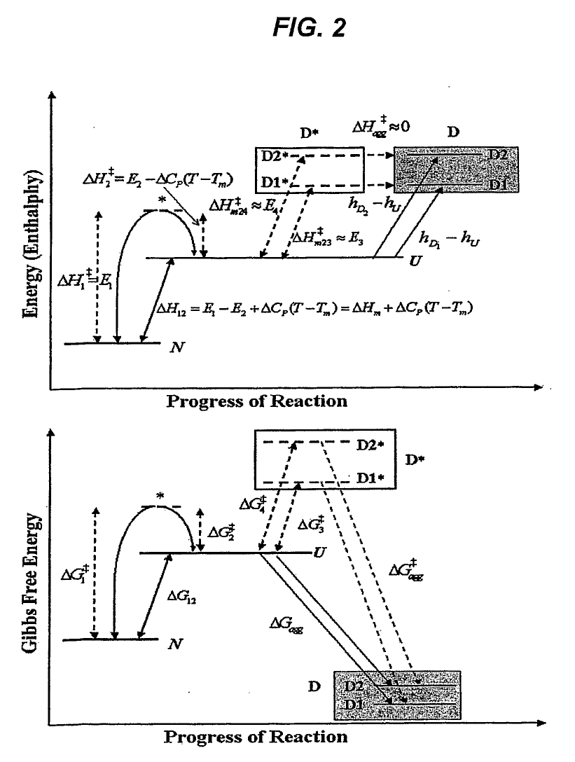

Previous published work pertaining to the fitting or simulation of DSC experiments were described by kinetic and thermodynamic contributions. Kinetic equations describe the rate of unfolding and aggregation reactions while the measured thermodynamic quantity is the excess heat capacity C_{P}. In the case of a two-state reversible system in a steady-state (see FIG. 2, where N is the average of the ensemble native state, and U is the average of the ensemble unfolded state), the thermodynamic part can be simply described by an equilibrium constant of the unfolding reaction,

$$\begin{matrix}{K}_{12} = exp ( - Δ G / ( RT ) ) \\ = exp ( Δ S / R - Δ H / ( RT ) )\end{matrix} \qquad ( 2 )$$

where ΔG, ΔS, and ΔH correspond to the change in Gibbs free energy, entropy, and enthalpy pertaining to the reaction. It is important to note that the thermodynamic parameter, K₁₂, can also be described in terms of the kinetic rate constants for the forward and reverse reactions as described in eqn (1). The measured quantity in the DSC experiment is the excess heat capacity,

$${C}_{P} ( T ) = ( Δ {H}_{m} + Δ {C}_{P} ( T - {T}_{m} ) ) ( - \frac{ⅆ N}{ⅆ T} ) + Δ {C}_{P} \cdot U ( T )$$

where the temperature increases linearly in time. ΔC_{P} is the change in heat capacity between the native and the denatured states. It has been ascribed to the exposure of hydrophobic surface to the solvent during thermal unfolding. Temperature may be described in terms of the scan rate ν (° C./min) in the expression T=T₀+νt where T₀ is the initial temperature of the scan.

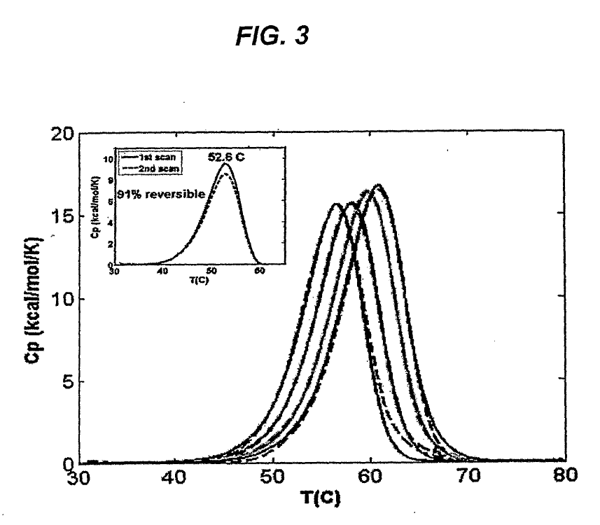

For a fully reversible system, thermodynamically meaningful parameters can be determined. In regard to rhuIL-1R(II), a fully reversible calorimetry experiment was nearly achieved when the protein concentration was 0.44 mg/mL in a solution consisting of 2 M urea (nondenaturing by CD at 20° C.) and 0.1 M sodium phosphate for buffering at pH 7 (see inlay of FIG. 3). The thermodynamic quantities measured in the fully reversible case are ΔHcal=82.5±2.5 kcal/mol, ΔCₚ=1.0±0.5 kcal/mol/K, and Tₘ=325.8±0.2° K. In this case the ΔH_{cal}/ΔHᵥₕ ratio was ˜0.9, (where ΔH_{cal} is the calorimetric and/ΔHᵥₕ is the van't Hoff enthalpies) suggesting the unfolding process was essentially two-state. Although urea can shift the Tm to lower temperatures and lower the enthalpy of unfolding, we have used non-denaturing levels of urea (as measured by CD at 20° C.) that should minimally perturb the unfolding transition, allowing the conditions used to represent an approximate reference point of the true Tₘ and associated thermodynamic parameters in the absence of the irreversible step.

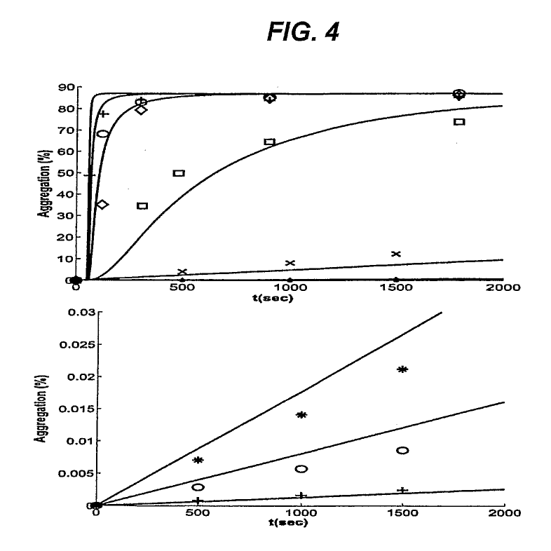

The experiment showed that urea effectively blocked the progress of aggregation, and that the system achieved 91% thermal reversibility in its presence. In the absence of urea, massive aggregation was observed at T>Tₘ (see FIG. 4), and the process was found to be predominantly irreversible. The DSC scan experiment also produced different behaviors, the most prominent being an increase in the apparent melting temperature with increasing scan rates in addition to a possibly negative influence in the ΔCₚ on the high temperature side of the unfolding envelope. An explanation for this phenomenon affiliated with aggregation has been reported previously.

Another important observation between the fully reversible and the partially irreversible thermal denaturation studies of rhuIL-1R(II) carried out in the DSC is the additional heat of the reaction making the total AH quantity higher than the urea experiment. This amounts to ˜48 kcal/mol more heat in the irreversible reaction approaching a total ΔH of 130 kcal/mol (FIG. 3). This would indicate that an additional endothermic contribution exists within the reaction process that involves the aggregate. It is proposed that this additional heat may arise from subsequent unfolding contributions resulting from protein-protein adsorption leading to the two D aggregated states of the model (as depicted in FIG. 2). Such endothermic heats have been observed for bovine milk a-lactalbumin where the subprocesses of sorbent (negatively-charged polystyrene latex), protein dehydration and protein denaturation contribute to the overall driving force of surface adsorption. In the case of rhuIL-1R(II), the sorbent is either another unfolded protein or soluble aggregate, and dehydration occurs by the removal of water from hydrophobic interfaces with the solvent, driving the reaction to the D states. In this process, a favorable increase in the entropy of the solution is expected resulting from hydrophobic surface area reduction as the aggregates continue to grow. In other words, the aggregation reaction of unfolded protein molecules in aqueous solution is an entropically driven reaction.

As for kinetics, the SEC data shown in FIG. 4 clearly indicate that there is a transition temperature where the aggregation rate of the system changes so that one cannot simply explain the denaturation process in terms of a single step described by an Arrhenius rate as in the work of Sánchez-Ruiz and coworkers. Nevertheless it is still a useful model to obtain an estimated activation energy. The expanded model described by Lepock et al. appears to be a better description of the system where all reactions are modeled as first order reactions.

However, our experimental observation of the concentration dependence of aggregation revealed that the approximate order of reaction was 1.70±0.04, suggesting a mixture of first and second order reactions participate in the system. Therefore, we add to Lepock's model an extra term describing the contribution of the second order processes. More specifically, we denote the aggregates resulting from the first order term as D₁, and those from the second order term as D₂. Together they are the irreversibly denatured population, D, with D₁* and D₂* representing the corresponding transition states that conceivably are aggregation competent species (see FIG. 2). The reaction can be represented by

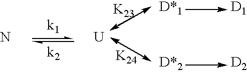

In the pathway from U [symbol] D₁* or U [symbol] D₂*, the reaction is assumed ₁^{st} order and reversible. From D₁*→D₁ and from D₂*→D₂ the reaction follows 2^{nd} order kinetics. Within the U [symbol] D₁*→D₁ path, the U→D₁* is rate limited and therefore, the overall reaction from U→D₁ is 1^{st} order. Likewise, in the path from U [symbol] D₂*→D₂, the D₂*→D₂ step is rate limiting and therefore the overall kinetics from U→D₂ is second order.

It should be noted in FIG. 4 that at the elevated temperature above 58° C. the reaction does not approach 100% aggregate. The data indicate a point of saturation that appears to approach 84% to 87%. This observation indicates that there is a remnant of protein that does not participate in the aggregation process. This remnant contribution is also observed in DSC thermal reversibility experiments and suggests that the approximate 13% of rhuIL-1R(II) can be accounted as fully reversible species in contrast to the majority of the molecules that go through the unfolding process leading to aggregates.

2. The Kinetic Equations and the Reaction Rates

Since the quantity or population of the transition state (i.e., D₁* and D₂*) are short lived, leading rapidly to the final states, D₁ and D₂, the kinetics of the system may be represented by

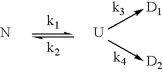

Assuming the total molar concentration of protein in the solution is [N₀], the molar concentration at each state can be normalized by this number to get a dimensionless relative concentration, i.e.,

$$\begin{matrix}N = \frac{[ N ]}{[ {N}_{0} ]} & ( 3 ) \\ U = \frac{[ U ]}{[ {N}_{0} ]} & ( 4 ) \\ D = \frac{[ D ]}{[ {N}_{0} ]} & ( 5 )\end{matrix}$$

It is important to realize that [D] represents the molar concentration of irreversibly denatured protein (aggregate weight concentration/monomer protein molecular weight). It is a collection of aggregate expressed as an equivalent portion of monomeric molecular forms.

The initial condition of the system is N(t=0)=1, U (t=0)=0 and D(t=0)=0. The differential equations describing the kinetics of the system are as follows:
\dot{N}=−k₁N+k₂U (6)
\dot{U}=k₁N−(k₂+k₃)U−k₄U² (7)
\dot{D}=k₃U+k₄U² (8)
where the U term results from the second order process and where D=D₁+D₂ with D₁ corresponding to the first order term physically representing the reaction of the unfolded molecule with a constant supply of aggregate molecules to form adducts and D₂ corresponds to the second order term depicting the formation of pseudo-steady-state dimmer leading to higher order aggregates. The terms k₁, k₂, k₃, and k₄ are the corresponding rate constants.

The second order reaction coefficient k₄ is necessarily proportional to the concentration [N₀], so that equations (6)-(8) hold for any [N₀]. The following derivation illustrates this conclusion: Eqn (7) is derived from the kinetic equation for the unnormalized concentration [U]:
[\dot{U}]=k₁[N]−(k₂+k₃)[U]−k₄′[U]² (9)
Dividing eqn (9) by [N₀] on both sides one obtains

$$\frac{[ \dot{U} ]}{[ {N}_{0} ]} = {k}_{1} \frac{[ N ]}{[ {N}_{0} ]} - ( {k}_{2} + {k}_{3} ) \frac{[ U ]}{[ {N}_{0} ]} = {k}_{4}^{′} [ {N}_{0} ] {( \frac{[ U ]}{[ {N}_{0} ]} )}^{2} . \qquad ( 10 )$$

Now by substituting in the definitions of eqns (3)-(5) one arrives at
\dot{U}=k₁N−(k₂+k₃)U−(k₄′[N₀])U². (11)
In order for eqn (11) to agree with eqn (7), it must be that k₄=k₄′[N₀],so that k₄′ denotes a linear rate proportionality constant.

The linear dependence of k₃ on [N₀] is not an obvious because it appears as a coefficient for a first order reaction rate. Since it originates from the reaction of one unfolded molecule with any aggregate, it is reasonable to assume the same linear relation to [N₀] holds true, and therefore both k₃ and k₄ can be expressed as
k₃=k₃′[N₀] (12)
k₄=k₄′[N₀] (13)
k₃′ denotes a linear rate proportionality constant analogous to k₄′. It is important to recognize that k₃ is proportional to the initial protein concentration.

The unfolding rate coefficient k₁ is known to be well described by the Arrhenius law:
k₁=exp (A₁−E₁/RT). (14)
Recalling the experiment with urea (see the inlay of FIG. 3), a positive change in the baseline molar heat capacity, ΔC_{P}, was observed. This implies that k₂ has non-Arrhenius behavior (see eqn (17)), as derived from the equilibrium constant of the folding/unfolding reaction and the modified Gibbs-Helmholz equation:

$$Δ {G}_{12} = Δ {H}_{m} ( 1 - \frac{T}{{T}_{m}} ) + Δ {C}_{P} [ T - {T}_{m} - T ln ( \frac{T}{{T}_{m}} ) ] \qquad ( 15 )$$

From this, one can write the corresponding equilibrium constant as

$$\begin{matrix}{K}_{12} = \frac{{k}_{1}}{{k}_{2}} \\ = exp ( - Δ {G}_{12} / ( RT ) ) \\ = exp ( - Δ {H}_{m} ( 1 - \frac{T}{{T}_{m}} ) + Δ {C}_{P} [ T - {T}_{m} - T ln ( \frac{T}{{T}_{m}} ) ] / ( RT ) )\end{matrix} \qquad ( 16 )$$

Substituting eqn (14) into eqn (16) and defining E₁-E₂≡ΔHₘ, and

$${A}_{1} - {A}_{2} ≡ \frac{Δ {H}_{m}}{{RT}_{m}}$$

$$( therefore {T}_{m} = \frac{{E}_{1} - {E}_{2}}{R ( {A}_{1} - {A}_{2} )} ) ,$$

one can solve for an expression of k₂,

$${k}_{2} = exp ( {A}_{2} - \frac{{E}_{2}}{RT} - \frac{Δ {C}_{P}}{R} [ \frac{{T}_{m}}{T} - 1 + ln ( \frac{T}{{T}_{m}} ) ] . \qquad ( 17 )$$

The above treatment has been well supported by experimental observations. Note that it is assumed that the ΔC_{P} here is approximately equal to the ΔCₚ measured in the experiment with urea and it is the only contribution that modifies Arrhenius rates. The term E₂ is the refolding activation energy when temperature equals Tₘ.

The kinetic rate constants, k₃ or k₄, can be determined according to Eyring's model and shown to conform to a similar expression of the form for k₂. Knowing that an equilibrium between the U and D₁* or U and D₂* is proposed to exist, there must be corresponding equilibrium constants K₂₃ and K₂₄ that describe this part of the system. These constants assume the same for as K₁₂ but replace the parameters {ΔHₘ, Tₘ, ΔC_{P}} with {ΔHₘ₂₃, Tₘ₂₃, ΔC_{P23}} for k₃ and {ΔHₘ₂₄, Tₘ₂₄, ΔC_{P24}} for k₄. Multiplying the resulting equilibrium constants in each case by kₓ ( kBT/2xh) (where the subscript x=3 or 4 corresponding to either k₃ or k₄; kₓ is the transmission co-efficient) Raising the multiplication factor to an exponent, it can be combined with the exponential form of eqn (16) to yield,

$${k}_{3} = exp ( \begin{matrix}[ \frac{Δ {H}_{m 23}}{{RT}_{m 23}} - \frac{Δ {C}_{P 23}}{R} ( 1 + ln {T}_{m 23} ) + ln \frac{{k}_{3} {k}_{B}}{2 πℏ} ] - \\ \frac{Δ {H}_{m 23} - Δ {C}_{P 23} {T}_{m 23}}{RT} + \\ \frac{Δ {C}_{P 23} + R}{R} ln T\end{matrix} )$$

$${k}_{4} = exp ( \begin{matrix}[ \frac{Δ {H}_{m 24}}{{RT}_{m 24}} - \frac{Δ {C}_{P 24}}{R} ( 1 + ln {T}_{m 24} ) + ln \frac{{k}_{4} {k}_{B}}{2 πℏ} ] - \\ \frac{Δ {H}_{m 24} - Δ {C}_{P 24} {T}_{m 24}}{RT} + \\ \frac{Δ {C}_{P 24} + R}{R} ln T\end{matrix} )$$

The resulting k3 and k4 expressions are grouped to show two temperature dependent terms and one temperature independent constant term. The temperature independent or constant term is enclosed within the brackets separated from the two temperature dependent terms in the exponential. Among the temperature dependent terms, one is inversely proportional to temperature and the other is proportional to 1n.T. Among the parameters, ΔHₘ₂ₓ, T_{m2x, ΔCP}_{2x, kx}, we have adopted the following defined relations that transform the expressions above into equations for k₃ and k₄ that are similar to eqn (17). For k₃, we define

$$Δ {C}_{P}^{{D}_{1}} ≡ Δ {C}_{P 23} + R , {E}_{3} - Δ {C}_{P}^{{D}_{1}} {T}_{m} ≡ Δ {H}_{m 23} - Δ {C}_{P 23} {T}_{m 23} and$$

$${A}_{3} ≡ [ \frac{Δ {H}_{m 23}}{{RT}_{m 23}} - \frac{Δ {C}_{P 23}}{R} ( 1 + ln {T}_{m 23} ) + \frac{Δ {C}_{P}^{{D}_{1}}}{R} ( 1 + ln {T}_{m} ) + ln \frac{{κ}_{3} {k}_{B}}{2 πℏ} ]$$

Similar relations for k₄ can be used to derive the final equations for the rate constants shown below,

$$\begin{matrix}{k}_{3} = exp ( {A}_{3} - \frac{{E}_{3}}{RT} + \frac{Δ {C}_{P}^{{D}_{1}}}{R} [ \frac{{T}_{m}}{T} - 1 + ln ( \frac{T}{{T}_{m}} ) ] & ( 18 ) \\ {k}_{4} = exp ( {A}_{4} - \frac{{E}_{4}}{RT} + \frac{Δ {C}_{P}^{{D}_{2}}}{R} [ \frac{{T}_{m}}{T} - 1 + ln ( \frac{T}{{T}_{m}} ) ] & ( 19 )\end{matrix}$$

Thus far, we have expressed the kinetic equations for the system in terms of a set of 11 independent parameters: {A₁, E₁, A₂, E₂, A₃, E₃, A4, E₄, ΔC_{P}, ΔC_{P}^{D}^{₁}, ΔCD_{P}^{D}^{₂} }. As will be shown in the next section, the excess heat capacity can also be expressed in terms of the same set of parameters. From these parameters Tₘ and ΔHm can be calculated.

3. The Observables: C_{P} and A_{gg}

Having described the kinetic model of the system, it was important to find expressions for the excess molar heat capacity C_{P} and the total mass of the aggregates during the reaction in terms of these kinetic parameters.

In the Time-Temperature Aggregation experiment, the total mass of the aggregates is proportional to 1−N(t)−U(t) or D. Here aggregation is defined by the expression,
A_{gg}(T, t)=D. (20)

It can be calculated by solving the differential eqns (6)-(8). From the aggregation equation (eqn (20)), one can derive an initial rate of aggregation as

$${R}_{0} ( T ) = \frac{ⅆ ( Agg ( T , t ) [ {N}_{0} ]}{ⅆ t} {\vert}_{t = 0} \approx ( Agg ( T , {t}_{1} ) / {t}_{1} * [ {N}_{0} ] \qquad ( 21 )$$

where t₁ is the first time point in the measurement for a given temperature. Agg(T,0) is assumed to be zero and [N₀] is the initial molar concentration of the protein.

In the DSC experiment, the measured quantity is the molar excess heat capacity under constant pressure.

$${C}_{P} = ( \frac{\partial h}{\partial T} ) P$$

where h is the total molar excess enthalpy of the system involved in the denaturation process. In the proposed model, the states involved in the endotherm are N, U, D₁, and D₂ with N=U=D₁+D₂=1.

$$\begin{matrix}h = {h}_{N} N + {h}_{U} U + {h}_{{D}_{1}} {D}_{1} + {h}_{{D}_{2}} {D}_{2} - {h}_{N} \\ = {h}_{N} N + {h}_{U} U + {h}_{{D}_{1}} {D}_{1} + {h}_{{D}_{2}} {D}_{2} - {h}_{N} ( N + U + {D}_{1} + {D}_{2} ) \\ = ( {h}_{U} - {h}_{N} ) U + ( {h}_{{D}_{1}} - {h}_{N} ) {D}_{1} + ( {h}_{{D}_{2}} - {h}_{N} ) {D}_{2}\end{matrix} \qquad ( 22 )$$

where hₓ, (x=N, U, D₁, D₂) are the enthalpies of the corresponding ensemble states, consisting of native, unfolded, and the final aggregate states of the first and second order components of the reaction.

Now one can write the observed C_{P} more explicitly as

$${C}_{P} ( v , T ) = ( {h}_{U} - {h}_{N} ) \frac{\partial {D}_{1}}{\partial T} + ( {h}_{{D}_{1}} - {h}_{N} ) \frac{\partial {D}_{2}}{\partial T} + \frac{\partial ( {h}_{U} - {h}_{N} )}{\partial T} U + \frac{\partial ( {h}_{{D}_{1}} - {h}_{N} )}{\partial T} {D}_{1} + \frac{\partial ( {h}_{{D}_{2}} - {h}_{N} )}{\partial T} {D}_{2} \qquad ( 23 )$$

Since the temperature is increased linearly in time for each scan rate ν, i.e. T=T₀+νt, the temperature derivatives can be expressed in terms of the time derivatives and the scan rate using the chain rule, e.g.

$$\frac{\partial N}{\partial T} = \frac{\partial N}{\partial t} \frac{\partial t}{\partial T} = \frac{1}{v} \dot{N}$$

The same applies to U, D₁, and D₂. Using eqns (6)-(8), the equation can be written as

$${C}_{P} ( v , T ) = ( {h}_{U} - {h}_{N} ) ( - \frac{1}{v} \dot{N} ) + ( {h}_{{D}_{1}} - {h}_{U} ) \frac{{k}_{3}}{v} U + ( {h}_{{D}_{2}} - {h}_{U} ) \frac{{k}_{4}}{v} {U}^{ 2} + \frac{\partial ( {h}_{U} - {h}_{N} )}{\partial T} U + \frac{\partial ( {h}_{{D}_{1}} - {h}_{N} )}{\partial T} {D}_{1} + \frac{\partial ( {h}_{{D}_{2}} - {h}_{N} )}{\partial T} {D}_{2} \qquad ( 24 )$$

The native state is the standard state of the protein at standard temperature and pressure and an energy reference point. (h_{U}−h_{N}) can be expressed in terms of the unfolding enthalpy and ΔC_{P} (see FIG. 2).
(h_{U}−h_{N})=ΔHₘ+ΔC_{P}(T−Tₘ)=E₁−E₂+ΔC_{P}(T−Tₘ), (25)
it then follows that

$$\frac{\partial ( {h}_{U} - {h}_{N} )}{\partial T} = Δ {C}_{p} \qquad ( 26 )$$

Similarly, with the assumption that no entholpy change takes place in D* to D transitions (h_{D}_{₁},−h_{U}) and (h_{D2}_{₁},−h_{U}) can be determined by the equilibrium constants K₂₃ and K₂₄ defined in the previous section. Using the van't Hoff equation,

$$\begin{matrix}\begin{matrix}( {h}_{{D}_{1}} - {h}_{U} ) = - R \frac{\partial ln {K}_{23}}{\partial 1 / T} \\ = Δ {H}_{m 23} + Δ {C}_{P 23} ( T - {T}_{m 23} ) \\ \approx {E}_{3} + Δ {C}_{P}^{{D}_{1}} ( T - {T}_{m} )\end{matrix} & ( 27 ) \\ \begin{matrix}( {h}_{{D}_{2}} - {h}_{U} ) = - R \frac{\partial ln {K}_{24}}{\partial 1 / T} \\ = Δ {H}_{m 24} + Δ {C}_{P 24} ( T - {T}_{m 24} ) \\ \approx {E}_{4} + Δ {C}_{P}^{{D}_{2}} ( T - {T}_{m} )\end{matrix} & ( 28 )\end{matrix}$$

consequently,

$$\begin{matrix}\begin{matrix}\frac{\partial ( {h}_{{D}_{1}} - {h}_{N} )}{\partial T} = \frac{\partial [ ( {h}_{{D}_{1}} - {h}_{U} ) + ( {h}_{U} - {h}_{N} ) ]}{\partial T} \\ = Δ {C}_{P}^{{D}_{1}} + Δ {C}_{P}\end{matrix} & ( 29 ) \\ \begin{matrix}\frac{\partial ( {h}_{{D}_{2}} - {h}_{N} )}{\partial T} = \frac{\partial [ ( {h}_{{D}_{2}} - {h}_{U} ) + ( {h}_{U} - {h}_{N} ) ]}{\partial T} \\ = Δ {C}_{P}^{{D}_{2}} + Δ {C}_{P} .\end{matrix} & ( 30 )\end{matrix}$$

Substituting eqn (25-30 into eqn (24), we have an expression for Cₚ in terms of scan rate and temperature,

$${C}_{P} ( v , T ) = ( Δ {H}_{m} + Δ {C}_{P} ( T - {T}_{m} ) ) ( - \frac{1}{v} \dot{N} ) + Δ {C}_{P} U + \frac{{k}_{3}}{v} ( {E}_{3} + Δ {C}_{P}^{{D}_{1}} ( T - {T}_{m} ) ) U + \frac{{k}_{4}}{v} ( {E}_{4} + {C}_{P}^{{D}_{2}} ( T - {T}_{m} ) ) {U}^{2} + ( Δ {C}_{P}^{{D}_{1}} + Δ {C}_{P} ) {D}_{1} + ( Δ {C}_{P}^{{D}_{2}} + Δ {C}_{P} ) {D}_{2} \qquad ( 31 )$$

The first line of eqn (31) contains the familiar terms describing the 2-state reversible system. The terms on the second line of eqn (31) are necessary to describe the contribution of the aggregation step to the overall heat absorption. When k₃ and k₄ are zero, so are D₁ and D₂, the equation reduces to the fully reversible case. When the sum, ΔC_{P}^{D}^{₁}+ΔC_{P} or ΔC_{P}^{D}^{₂}+ΔC_{P} do not equal zero, the last two terms give a non-vanishing baseline shift that can contribute to a negative net influence on the transition baseline. The negative influence on the transition baseline can occur when ΔC_{P}^{D}^{₁} or ΔC_{P}^{D}^{₂} result from buried hydrophobic surface as would be expected in the aggregation process. After subtraction of the transition baseline, the last two terms in eqn (31) are of no consequence. Therefore, the fitted data shown in FIG. 3 are based on this equation with the last two terms excluded.

III. Applications

Both methods and computer apparatus for implementing the new approach will be described below.

A. Example Method

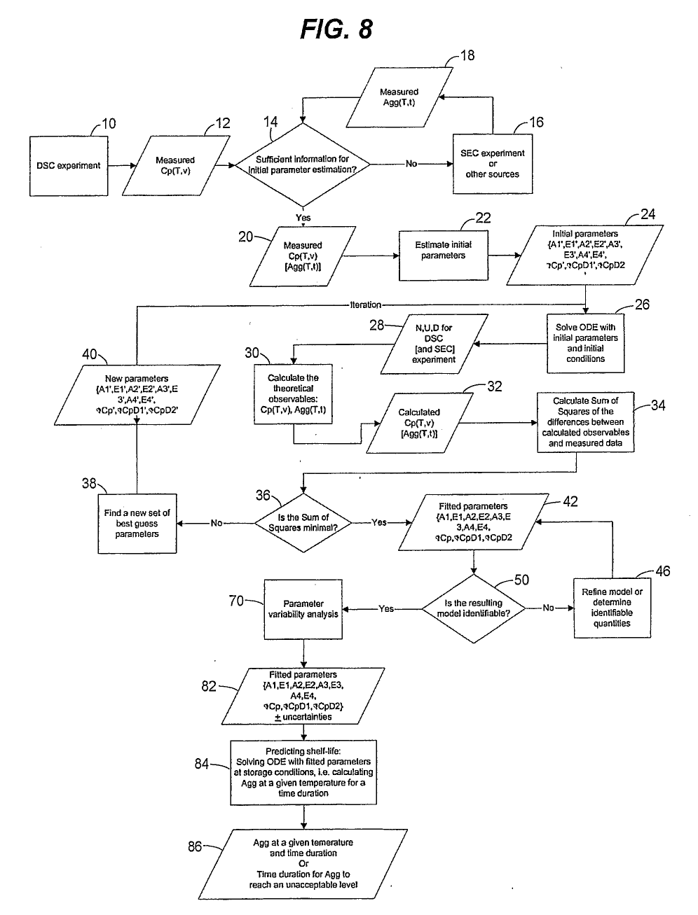

An example of the basic steps of providing measurements of conformational change, using those measurements to rigorously derive useful parameters, and using the parameters is set forth in FIG. 8, and is described below.

1. Providing Measurements of Conformational Change

The methods of data acquisition will be discussed after a description of some of the various materials in connection with which the method can be used.

a. Compositions

The methods of the invention can be used with any biologically active material that is capable of a conformational change and, in certain embodiments, unfolding, due to a thermal change and that exhibits multi-state behavior provided that such behavior can be measured by a suitable biophysical procedure as described further below. In certain embodiments wherein the material exhibits behavior that has more than two states, the behavior is dissected into two-state segments.

The biologically active material may be a polypeptide or protein. Such materials include but are not limited to hormones, cytokines, hematopoietic factors, growth factors, antibodies, antiobesity factors, trophic factors, anti-inflammatory factors, and enzymes (see also U.S. Pat. No. 4,695,463 for additional examples of useful biologically active agents).

The material could also include antibodies, antibody-like molecules, interferons (see, U.S. Pat. Nos. 5,372,808, 5,541,293 4,897,471, and 4,695,623 hereby incorporated by reference including drawings), interleukins (see, U.S. Pat. No. 5,075,222, hereby incorporated by reference including drawings), erythropoietins (see, U.S. Pat. Nos. 4,703,008, 5,441,868, 5,618,698 5,547,933, and 5,621,080 hereby incorporated by reference including drawings), granulocyte-colony stimulating factors (see, U.S. Pat. Nos. 4,810,643, 4,999,291, 5,581,476, 5,582,823, and PCT Publication No. 94/17185, hereby incorporated by reference including drawings), stem cell factor (PCT Publication Nos. 91/05795, 92/17505 and 95/17206, hereby incorporated by reference including drawings), and leptin (OB protein) (see PCT publication Nos. 96/40912, 96/05309, 97/00128, 97/01010 and 97/06816 hereby incorporated by reference including figures).

The materials might also include substances like insulin, gastrin, prolactin, adrenocorticotropic hormone (ACTH), thyroid stimulating hormone (TSH), luteinizing hormone (LH), follicle stimulating hormone (FSH), human chorionic gonadotropin (HCG), motilin, interferons (alpha, beta, gamma), interleukins (IL-1 to IL-12), tumor necrosis factor (TNF), tumor necrosis factor-binding protein (TNF-bp), brain derived neurotrophic factor (BDNF), glial derived neurotrophic factor (GDNF), neurotrophic factor 3 (NT3), fibroblast growth factors (FGF), neurotrophic growth factor (NGF), bone growth factors such as osteoprotegerin (OPG), insulin-like growth factors (IGFs), macrophage colony stimulating factor (M-CSF), granulocyte macrophage colony stimulating factor (GM-CSF), megakaryocyte derived growth factor (MGDF), keratinocyte growth factor (KGF), thrombopoietin, platelet-derived growth factor (PGDF), colony simulating growth factors (CSFs), bone morphogenetic protein (BMP), superoxide dismutase (SOD), tissue plasminogen activator (TPA), urokinase, streptokinase, and kallikrein.

In certain embodiments, the biologically active material will be formulated as a pharmaceutical composition comprising effective amounts of the biologically active material, or derivative products, together with pharmaceutically acceptable excipients, for example, diluents, preservatives, solubilizers, emulsifiers, adjuvants, and/or carriers needed for administration. The optimal pharmaceutical formulation for a desired biologically active material can be determined by one skilled in the art depending upon the route of administration and desired dosage. See, e.g., Remington's Pharmaceutical Sciences (Mack Publishing Co., 18th Ed., Easton, Pa., pgs. 1435-1712 (1990)).

The material could also be a solution that could be in either liquid or solid amorphous state, or in a glassy state. Sometimes, the material may have been lyophilised.

b. Data Acquisition

Conformational change (e.g., unfolding) of these compositions can be measured as a function of temperature varied with time. In some case, it may be important for temperature to be varied uniformly with time.

A variety of different thermodynamic parameters can be determined, including: enthalpy of the transition (ΔH) or the free energy (ΔG); ΔCₚ (the change in heat capacity between native and denatured states of the material); and the temperature at which about 50% of the molecule is unfolded and about 50% of the molecule is in its native state (for example, Tₘ, T_{g}, or Cₘₐₓ).

When the biologically active material is a nucleic acid, the melting temperature of the nucleic acid can be measured. In other cases, the hybridization properties of the nucleic acid can be analyzed.

Differential scanning calorimetry or DSC can might be used to determine the temperature of denaturation if the system is fully reversible and the scan rate does not exceed the rate of unfolding. See, Privalov, P. L.; Khechinashvili, N. N. J. Mol. Biol. 1974, 86, 665-684 and Lepock, J. R.; Ritchie, K. P.; Kolios,.M. C.; Rodahl, M; Heinz K. A.; Kruuv, J. Biochemistry 1992, 31, 12706-12712. In other embodiments, circular dichroism or CD can be used to analyze the composition. The measured quantity in the CD experiment is molar ellipticity.

Fluorescence can sometimes be used to analyze the composition. Fluorescence encompasses the release of energy in the form of light or heat, the absorption of energy in the form or light or heat, changes in turbidity, and changes in the polar properties of light. Specifically, the term refers to fluorescent emission, fluorescent energy transfer, absorption of ultraviolet or visible light, changes in the polarization properties of light, changes in the polarization properties of fluorescent emission, changes in turbidity, and changes in enzyme activity. Fluorescence emission can be intrinsic to a protein or can be due to a fluorescence reporter molecule. For a nucleic acid, fluorescence can be due to ethidium bromide, which is an intercalating agent. Alternatively, the nucleic acid can be labeled with a fluorophore.

HPLC methods can also sometimes be used to analyze the conformational change of the biologically active material.

In this example, we began with the DSC experiment (block 10 in FIG. 8), which provided measured data for C_{P} (T,ν) (block 12). Before proceeding, we evaluated whether we had enough information for making a good initial estimate of the initial parameters (14). In this case, we wanted both to use C_{P} (T,ν) and A_{gg} (T,t) for estimating the initial parameters. Accordingly, we obtained further data from other sources. We used an SEC time aggregation study (16) to provide aggregation data (18), giving us data on both C_{P} (T,ν) and A_{gg} (T,t) (block 20).

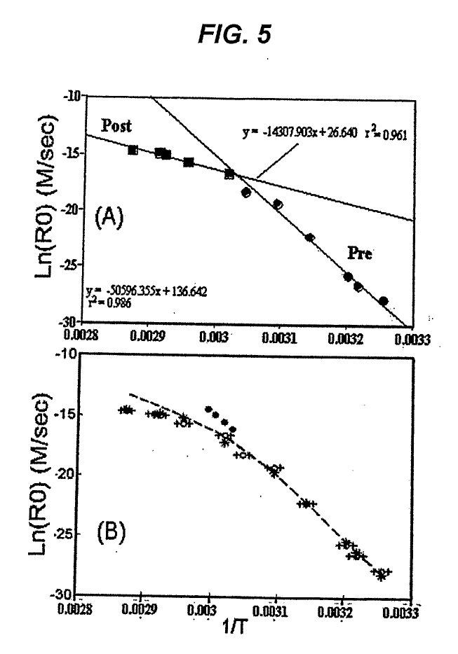

The rate of aggregation was derived from a plot showing the amount of aggregate measured by SEC as a function of time as depicted in FIG. 4 using eqn (21). At low temperatures between 30 and 50° C., linear rates of reaction were easily ascertained since there was no deviation from linearity during the period of time examined. However, at temperatures >58° C., the initial reaction rate was calculated according to eqn (20). The observed rate constants obtained from these data were converted to units of M/sec and then subsequently evaluated in the form of an Arrhenius plot (FIG. 5A).

It has been shown that it is possible to fit the data with two lines, reflecting approximate Arrhenius behaviour above and below the apparent melting temperature (as depicted in FIG. 5A). The data points that determine the lines were found by optimizing both correlation coefficients. From the slope of the lines describing these two regions, one is able to characterize the aggregation reaction kinetics with two activation energies. At low temperatures (below Tₘ), an activation energy of 100 kcal/mol was obtained and at high temperatures (above Tₘ), the activation energy was found to be approximately 28 kcal/mol.

The Arrhenius data in FIG. 5A clearly show a break from linearity in the reaction rate around the melting temperature, indicating two different activation energies at two different temperature regions, separated approximately by Tₘ. This behavior can be explained by a rate approximation from the kinetic model (eqn (6)-(8)). When k₃<<k₂ and k₄<<k₃ (valid at low temperatures below the Tₘ), the equilibrium between N and U predominates as expected and the aggregation can be described the an effective rate coefficient

$${k}^{′} = \frac{{k}_{1} {k}_{3}}{{k}_{1} + {k}_{2} + {k}_{3}} .$$

The effective rate is plotted as the dashed line in FIG. 5. It can be seen that this approximation is very good at low temperature when (T<<Tₘ, k₂/k₁>>1 therefore k′≈k₃k₁/k₂). When T >>Tₘ, k′≈k₃, although the rate approximation for aggregates is not as good in this temperature region, one can still obtain a qualitative estimate of k₃ from the experimental data. It should be noted that our numerical fitting was performed over the entire time-sequence of study and not just limited to the initial rates. The initial rates are used to estimate the starting parameters for the nonlinear fitting and used for checking the consistency of the final results.

In the DSC experiment, the apparent melting temperature Tₐₚₚ depends on the scan rate of the irreversible system. The scan-rate dependent activation energy of the Tₐₚₚ was also examined to ascertain the relevance to the activation energy obtained from the Arrhenius plot of the time-temperature studies. The results obtained from a plot of In └ν/Tₐₚₚ²┘ as a function of 1/T as proposed by Sánchez et.al. yielded a straight line with an associated activation energy of about 89 kcal/mol. This treatment appears to yield an activation energy that is consistent with the low temperature fit in the SEC experiment. Namely, a respectable result that compares well with the activation energy obtained from the time-temperature studies.

For illustration purposes, the scan-rate method of Sánchez-Ruiz and coworkers is plotted as solid circles in FIG. 5B [within the region from 0.0029 (61.3° C.) to 0030 (56.9° C.)]. The lower activation energy of the scan-rate dependent result may exhibit some bias imposed by the region of curvature so that extrapolation to commercially important low temperatures (i.e., 2-8° C.) would result in overestimates of aggregation reaction rates.

2. Obtaining Parameters

As discussed below, useful parameters can be rigorously obtained by making an initial estimate, fitting the data to obtain fitted parameters, checking the uniqueness of those parameters, and then assessing their variability.

a. Making Initial Estimates

The measured A_{gg} (T,t) data is shown in FIG. 1. Together, C_{P} (T,v) and A_{gg} (T,t) enabled one of skill in the art to make an initial rough estimate of the parameters of interest (blocks 22, 23 in FIG. 8). Those initial rough estimates were used as the starting point for fitting the data.

b. Fitting the Data

Exemplary parameter estimation methods that can be used to compare predicted measurements of conformational change to actual measurements of conformational change include non-linear least squares fitting method, maximum likelihood method, non-linear least norms method, and other methods known in the art.

In this example, eqn (31) was used to fit the experimental data to extract all the Arrhenius parameters. In general, the original differential equations were solved with initial parameters and initial conditions (block 26 in FIG. 8), giving N, U, and D for the experiments (28). From these, theoretical values of Cp(T,v) and Agg(T,t) were calculated (30, 32). These calculated values were compared to the measured data, and the sum of the squares of the differences were calculated (34). The sum was evaluated to determine if it was minimal (36). When it was not minimal, a new set of best guess parameters was created (38, 40) and a new iteration began, the process continuing until the sum of squares was minimal.

In this case, a nonlinear least square fitting routine (Isqnonlin in Matlab) was applied to minimize a linear combination of the two sum-of-squares functions simultaneously. The first represents the difference between the calculated values and the observed values in the DSC experiment, and the second represents the difference between the calculated and observed values in the SEC experiment. Denoting the calculated values with a superscript calc and the observed values with a superscript exp, the two sum of squares functions can be written as,

$${χ}_{1} = {\sum }_{i = 1}^{4} {\sum }_{i = 1}^{{n}_{i}} {w}_{i} {\vert {C}_{P}^{calc} ( {v}_{i} , {T}_{j} ) - {C}_{P}^{exp} ( {v}_{i} , {T}_{j} ) \vert}^{2} \qquad ( 32 )$$

where νᵢ, (i=1, 2, 3, 4) is the designated scan rate and nᵢ, (i=1, 2, 3, 4) represents the corresponding data points of each scan. The term wᵢ, is an optional weighting factor to ensure each data point is counted appropriately. The reason for the weighting factor is to account for the collection of more data points at the slower scan rate than at the faster scan rate. Without weighting, each data point contributes equally to the sum-of-squares function. This leads to more contributions from the slower scan data. The weighting factor of wᵢ=1/nᵢ makes the contribution of each scan equal.

$${χ}_{2} = {\sum }_{i = 1}^{9} {\sum }_{j = 1}^{{n}_{i}} {\vert ln ( {Agg}^{calc} ( {T}_{i} , {t}_{j} ) ) - ln ( {Agg}^{exp} ( {T}_{i} , {t}_{j} ) ) \vert}^{2} \qquad ( 33 )$$

where i=1 to 9 represents the 9 temperatures under which the aggregation experiment took place. We used nᵢ=3, i.e. the first three points at each temperature to do the fitting.

Note that either x₁ or x₂ can be used alone for fitting the parameters. In fact that is what was often done in the literature. But the sensitivity of the two observed quantities to each parameter is different. In order to best constrain the parameter space, we performed a simultaneous fitting for both. In other words we minimized the following quantity:
χ=χ₁+F·χ₂ (34)
where F is an arbitrary scaling factor selected to make contributions from χᵢ, and F·χ₂ to χ similar in order of magnitude, since the two quantities have different dimensions and would be otherwise incomparable.

After performing the procedure of the nonlinear least square fitting, we obtained numerical values for all the parameters in the model system. As discussed above, since the aggregation reaction is entropically driven, the transition from D₁* and D₂* to the final D₁ and D₂ states was spontaneous, driven by the removal of hydrophobically exposed surfaces during aggregation. Little or no heat was released ΔH_{agg}^{‡}≈0) in this process, but the entropy of the solution was increased. So the change in Gibbs free energy ΔG_{agg}^{‡} (see FIG. 2) can be expressed as

$$\begin{matrix}Δ {G}_{agg}^{‡} \approx - T Δ {S}_{agg}^{‡} \\ = - T ( Δ {S}_{{D}_{1}^{*} {D}_{1}}^{‡} \cdot {D}_{1} + Δ {S}_{{D}_{2}^{*} {D}_{2}}^{‡} \cdot {D}_{2} )\end{matrix} \qquad ( 35 )$$

The relevant parameters are displayed in Table 1.

TABLE 1
Parameters obtained from fitting the model to both DSC traces and
SEC aggregation curves simultaneously.

| $\begin{matrix}{A}_{1} \\ ln ( \frac{1}{min} )\end{matrix}$ | $\begin{matrix}{E}_{1} \\ \frac{kcal}{mol}\end{matrix}$ | $\begin{matrix}{A}_{2} \\ ln ( \frac{1}{min} )\end{matrix}$ | $\begin{matrix}{E}_{2} \\ \frac{kcal}{mol}\end{matrix}$ | $\begin{matrix}{A}_{3} \\ ln ( \frac{1}{min} )\end{matrix}$ |
|---|---|---|---|---|
| 115.4 ± 1.7 | 76.6 ± 1.2 | 1.0 ± 11.7 | 2.3 ± 8.0 | 68.9 ± 6.9 |

| $\begin{matrix}{E}_{3} \\ \frac{kcal}{mol}\end{matrix}$ | $\begin{matrix}{A}_{4} \\ ln ( \frac{1}{min} )\end{matrix}$ | $\begin{matrix}{E}_{4} \\ \frac{kcal}{mol}\end{matrix}$ | $\begin{matrix}{ΔH}_{m} \\ \frac{kcal}{mol}\end{matrix}$ |
|---|---|---|---|
| 46.8 ± 4.4 | 228.1 ± 22.6 | 151.7 ± 15.0 | 74.3 ± 6.8 |

| $\begin{matrix}{T}_{m} \\ K\end{matrix}$ | $\begin{matrix}ΔCp \\ \frac{kcal}{mol \cdot K}\end{matrix}$ | $\begin{matrix}{ΔC}_{P}^{{D}_{1}} \\ \frac{kcal}{mol \cdot K}\end{matrix}$ | $\begin{matrix}{ΔC}_{P}^{{D}_{2}} \\ \frac{kcal}{mol \cdot K}\end{matrix}$ |
|---|---|---|---|
| 326.6 ± 1.4 | 1.3 ± 0.7 | −0.2 ± 0.5 | −5.8 ± 1.6 |

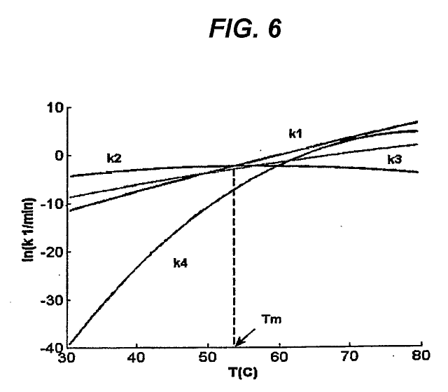

The rate constant profiles as a function of temperature are shown in FIG. 6 with corresponding activation energies in Table 1. Considering conditions below Tₘ in FIG. 6, the model depicts k₄ (with associated activation energy, E₄) as least important based on a slower reaction rate and correspondingly high-energy barrier (152 kcal/mol). E₃ on the other hand with associated rate constant k₃ has a lower activation energy (47 kcal/mol) than either E₁ or E₄, and therefore predominates the aggregation pathway, rate limited only by the supply of aggregation competent spaces D₁ and unfolded forms of the protein. This is explicit from the fact that k₁ is rate limiting and slower than k₃. Hence at temperatures below the Tₘ, solution conditions that tend to stabilize the native state will greatly impede formation of aggregate by either E₃ or E₄ routes of aggregation.

From the post-Tₘ data fit of FIG. 5A, an activation energy of 28 kcal/mol was obtained. This activation energy was ascribed to a change in aggregation rate that depended upon unfolding. Although tempting to assert this 28 kcal/mol activation energy to E₃ as described by the Lepock model, we have found that the theory contained in our model presents a more complex picture of the transition states post-unfolding. As temperature exceeds the Tₘ, k₄ becomes dominant overtaking k₃ even though the activation energy barrier is greater than E₃. It is important to realize that the U state is sufficiently populated to supply both D₁* and D₂* at or above the Tₘ. The reaction pathway driven by k₃ and k₄ are no longer rate limited by k₁. Hence the rate, k₃, becomes less prominent, while k₄ becomes the rate determining factor post-Tₘ as described in FIG. 6 even though its activation energy is greater than k₃. The result leads to a reasonable fit of the data as depicted in FIG. 5B.

It can be seen from FIG. 3 that there is good agreement between eqn (31) and the experimental data. It can be seen that they span a range of 10 orders of magnitude. For that reason, a system of stiff differential equations (eqns 6-8) needs to be solved. Matlab ODE Solver ode15s was used. The temperature where k₁ and k₂ cross corresponds to the Tₘ (˜53.5° C.) for a fully reversible unfolding system that involves only the native and unfolded states.

The comparison of calculated aggregation based on the model and the experimentally obtained SEC data is shown in FIG. 4 and FIG. 5B. Note the temperature associated with each data point has a variability of ±1° C. as shown in the plot. Regarding the calculated rates shown in FIG. 4, the curves above Tₘ are calculated from an initial condition N(t=0)=1, U(t=0)=D₁(t=0)=D₂(t=0)=0 assuming a linear heating time of 60 sec from T₀=25° C., while the curves below Tₘ were calculated with an equilibrium between N and U at t=0, i.e. N(t=0)/U((t=0)=k₂/k₁.

TABLE 2
Comparison of initial rate predicted at 34° C. for the 1 mg/mL solution by
differing methods. For pre-Tₘ fit, see FIG. 5A. The rate for this model is
calculated by converting A_{gg} (T, t₁)/t₁ * [N₀] to the correct units.

| | Experiment | Sánchez-Ruiz | Pre-Tₘ fit | eqn (21) |
|---|---|---|---|---|
| rate(M/sec) | 8.5 · 10⁻¹³ | 3.8 · 10⁻¹² | 6.3 · 10⁻¹³ | 6.2 · 10⁻¹³ |

A comparison of the initial rate obtained from the Time-Temperature experiment at 34° C. to the rates obtained by three different methods is listed in Table 2. The aggregation rate predicted by the model (eqn (21)) and the pre-Tₘ fit best represents the experimentally determined aggregation rate in comparison to the Sánchez-Ruiz model.

There is another check for the consistency of all our parameters: the total heat (enthalpy) of the reaction. The experimental measurement can be directly calculated by:

$$Δ H = {\int }_{{T}_{0}}^{{T}_{F}} {C}_{P} ⅆ T \qquad ( 36 )$$

where T₀ and T_{F} are the beginning and final temperatures of the transition envelope. From eqn (31), we can obtain approximate expressions for this quantity:
ΔH≈(E₁−E₂)+E₃·D₁+2E₄·D₂ (37)

where E₁−E₂=ΔHₘ, the unfolding reaction enthalpy. D₁ and D₂ are obtained at the end of our numerical integration of eqn (6)-(8). The comparison is shown in Table 3. It can be seen that there is good agreement in ΔH between the experimental and calculated results.

TABLE 3
Comparison of total enthalpy measured (top row), approximation from
eqn (37) (middle row) and the simulated values from eqn (31)
(bottom row).

| υ (degree/min) | 0.25 | 0.5 | 1.0 | 1.5 | |
|---|---|---|---|---|---|
| eqn (36) | 128 | 129 | 138 | 130 | kcal/mol |
| eqn (37) | 123 | 131 | 141 | 147 | kcal/mol |
| eqn (31) | 125 | 131 | 138 | 140 | kcal/mol |

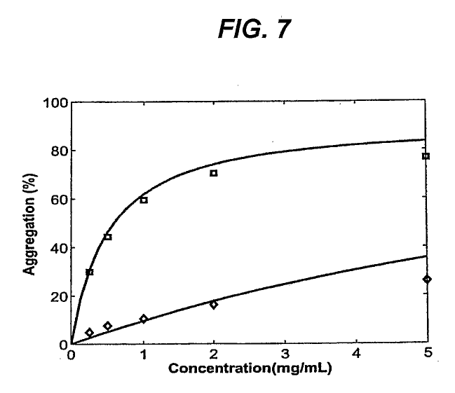

All the experimental data discussed so far were obtained with a fixed concentration of 2 mg /mL. Interestingly, the model can be applied to describe the concentration dependence of the aggregation rate (see eqns (12)-(13)). FIG. 7 shows the predicted and the experimentally determined concentration response at two different temperatures corresponding to different time durations. It shows the simple assumption that k₃ depends linearly on the concentration works fairly well at the lower concentration range but tends to overestimate aggregation rates at higher concentrations. As for why it deviates from the experimental results more significantly at the highest concentrations tested is unclear. A possible explanation may be that at higher concentrations there is a greater tendency for the protein to self-associate in the solution phase. Such molecular crowding as the protein concentration is increased can lead to an augmentation of aggregation. However, a portion of non-covalent self-associated aggregates can be reversible entities in solution that are not picked up by the SEC method and therefore may be observed lower than what actually exists in solution. This could occur as a result of dilution or during passage through the size exclusion column. The disparity between aggregation in solution and that determined by SEC would then be expected to increase with concentration where the theoretical prediction exceeds SEC aggregation results.

c. Checking the Uniqueness

We paid special attention to the question of identifiability: the ability to guarantee that all eleven parameters
P={A₁, E₁, A₂, E₂, A₃, E₃, A_{4, E}₄, ΔC_{P},ΔC_{P}^{D}^{₁}, ΔCₚ^{D}^{₂}}
are uniquely determined by minimizing eqn (34). (If the parameters were nonidentifiable, then different values for the eleven parameters could produce the same sum of squares.) (Consequently, Tₘ and ΔHm are also identifiable with reasonable variances.) It is not possible to guarantee this globally, but we can guarantee it locally near the calculated parameters p₀ yielding the minimum of χ (eqn (34)).

One possible way to assess model identifiability is laid out in FIG. 9. In that routine, there is first a symbolic calculation of the system of variational differential equations (block 52). Then there is a numerical solution of the variational differential equations at the values of the fitted parameters (54). Then a linearization of the mapping of the parameters to the observables at the values of the fitted parameters is built (56). This can be done, for example, by solving variational differential equations. Then a decision is made if the linearization is nondegenerate (58). If it is, then the system is identifiable (60). If not, then the width of the preimage of the unit cube in the functional space is checked to see if it is not infinity (62). If so, the system is identifiable (60). If not, the system is not identifiable (64).

We used variational methods to calculate the partial derivatives

$$\frac{\partial {C}_{P} ( {T}_{i} )}{{\partial }_{Pj}}$$

for each of the 11 parameters j=1, . . . , 11 at each of the n temperature points Tᵢ, i=1, . . ., n evaluated by the symbolic and numerical differential equation solver (in our calculations n was on the order of 200). This yielded a matrix of size 11 by n. Numerical calculation showed that the rank of this matrix was maximal size of 11, assuring the nondegeneracy of the defining equations and the identifiability of the parameters, so the values we calculated are indeed uniquely determined.

The numerical calculations showing that the rank of the matrix is maximal possible can be produced in different ways. For example it can be done using norms in the space of parameters and in the space of observables. The norm is a function that relates to each vector in the space a length of the vector.

One can introduce a norm (denote it by NO) in the space of the observables and a norm (denote it by NP) in the space of the parameters. Consider the subset of all vectors with the length equal to 1. This subset is called the unit subset. Denote the unit subset in the space of the observables by B1. The unit subset is given by the formula B1={o:NO(o)=1}. Consider the preimage of the unit subset B1 in the space of observables under the mapping ∂pC. Denote this preimage by prmg(B1). The preimage is given by formula prmg(B1)={p: NO(∂pC(p))=1}. The size of prmg(B1) is defined as the minimal number r such that the subset {p:NP(p)<=r} contains prmg(B1). Denote the size by R. Matrix ∂pC has maximal rank if and only if R is less than infinity. In the case when NO and NP are quadratic forms, R is equal to 1/svd where svd is the minimal singular value in singular values decomposition of the matrix ∂pC. It is also useful to use other norms NO and NP that give more precise evaluation of the invertibility of the matrix ∂pC because svd could be (and in fact is) very small for a particular case. For example, the norm of maximum of values of the components of parameters and maximum values of the components of the observables has been used. The size of the prmg(B1) for these norms is called the width of the prmg(B1).

d. Assessing Variability

Parameter variability (block 30 in FIG. 8) an be analyzed by the routine set out in FIG. 10. As shown there, we first determined the subspace of the functions to which errors belong, then projected the errors on the space of the observables (block 30 in FIG. 10), and found the 95% confidence area (the area in the projected set to which 95% of errors belong). We then found a preimage of the 95% confidence set using the linearization of the mapping from the parameters to the observables (32 in FIG. 10), and found widths of the preimage (34 in FIG. 10). Those widths established the 95% confidence interval.

3. Using the Parameters

A major hurdle to overcome is the ability to make reasonable estimates of aggregation half-life at conditions of low temperature storage. Storage conditions of marketed liquid biopharmaceuticals normally require refrigerated temperatures.

Most of the approaches used in this context have relied upon empirical modeling methods. Although these methods have applied Arrhenius models to make predictions about shelf-life, they have ignored the thermodynamic properties of reactions that can often result in non-Arrhenius behavior. In the study presented, a mechanistic model has been proposed to predict properties of shelf-life as it pertains to aggregation. The commercial viability of a drug product presented to health care personnel generally requires a minimum shelf life of eighteen months when stored at room temperature (20-25° C.) or under refrigeration (2-8° C.). The shelf life is typically defined as the storage time after manufacture of the drug product during which the drug experiences no more than about 5 to 10% degradation, i.e., the drug retains its biological activity.

This model has been applied to better evaluate non-Arrhenius reaction rates of rhuIL-IR(II) aggregation mediated by unfolding. In this first step taken, the validity of the simulation in regard to appropriately predicting the influence of concentration factors, deriving respectable thermodynamic parameters from a partially irreversible process, and emphasizing protein unfolding as a prerequisite to aggregation has successfully explained the aggregation kinetics of the system.

The thermal unfolding enthalpy in the absence of urea exhibited more heat (˜48 kcal/mol) than could be accounted for in the reversible case (with urea). We have considered other alternative modeling schemes to explain these results like different unfolded or U states where the enthalpy of urea was lower than the enthalpy in its absence (as dictated by the experimental results). Although one could still obtain a fit, it was not as good as the proposed model and there were other issues of conflict. For example in the case of altered unfolded states, ΔH≠E₁−E₂, and the expectation that it should be a valid equality has been suggested by Lepock and coworkers. Additionally, when k₃ and k₄ were made equal to zero (as in the fully reversible case), the theoretical Tₘ was ˜65° C. (above the highest scan Tₘ), a condition that does not satisfy and is far removed from the fully reversible case in urea (˜52.6° C.). Furthermore, the heat gained does coincide with the population of states≧Tₘ where massive aggregation has been confirmed in both the “Time-Temperature” SEC studies as well as those studies conducted in the calorimeter (in addition to the presence of a deconvoluted peak at ˜60° C. under the unfolding transition on the high temperature side that is close to the 48 kcal/mol enthalpy increase). In contrast, by adopting the theory as presented here, there was found greater harmony among the experimental observations (i.e., better fit of the data), where the 48 kcal/mol increase in enthalpy could be assigned to a deconvoluted peak on the high temperature side representing contributions from the D states (D₁ and D₂). Moreover, ΔHₘ more appropriately agreed with the E₁-E₂ equality (˜74.3 kcal/mol instead of ˜54 kcal/mol; closer to the 85 kcal/mol in the urea case). When k₃ and k₄ were set to zero, the Tₘ was approximately ˜54° C., much closer to the value in urea, but slightly higher as would be anticipated when chemical denaturant is absent. We examined the case where only a 2^{nd} order aggregation process was simulated and found an unfavorable ΔHₘ of ˜52 kcal/mol and a predicted Tₘ of 51° C. These values are not valid since they were not consistent with the measured enthalpy and Tₘ values in the DSC experiment using urea.

The corresponding transition states (D₁* and D₂*) of the D state are inferred from the observation of a noticeable change in kinetics above the Tₘ. This temperature zone coincides with more rapid aggregation kinetics (massive) than what was observed at temperatures below the Tₘ. It testifies to the validity of a constant supply of unfolded protein that can rapidly interact by either a 1st or 2nd order mechanism. Furthermore, justification for E₄ is found by the quality of fit on the high temperature side of the DSC endotherm.

The relationship between activation energy and total enthalpy of the unfolding transition described by Sánchez-Ruiz et al and Lepock et al was found inadequate as an appropriate description of the DSC behavior at rhuIL-1R (II). Moreover, they did not include influences of the ΔC_{P} that contribute to non-Arrhenius aggregation responses. Although the ΔC_{P} does not contribute significantly within the temperature regime in the vicinity of the Tₘ, it can pose more influence at lower temperature. In contrast to these approaches, the present work has derived a theoretical treatment that can be applied to DSC data in order to extract thermodynamically meaningful parameters from a partially reversible system. Unlike the work of Sánchez-Ruiz et al., and Lepock et al., this work describes the system in terms of both 1st and 2nd order reaction properties that depend upon the thermodynamics of unfolding. It takes into account the influences of the denaturational heat capacity in describing the non-Arrhenius kinetics of aggregation that can occur at low as well as high temperatures. Finally, it satisfactorily describes the enthalpy and activation energies along the aggregation reaction pathway and lays the groundwork for predicting shelf-life of complex protein aggregation systems.

The procedure described in the flow chart produced fitted parameters: {A1, E1, A2, E2, A3, E3, A4,E4, ΔCp, ΔCpD1, ΔCpD2}±uncertainties.

From these parameters we can calculate the kinetic rate constants at a given temperature, for example, 2° C., 4° C. and 8° C., using eqn (14), eqn (17), eqn (18) and eqn (19).

Substituting these constants into equations (6)-(8). Assuming the initial conditions, N(t=0)=1, U(t=0)=0, D1(t=0)=0, D2(t=0)=0, one can solve the differential equations (6)-(8) to obtain

1. For a given time duration, typically 2 years for liquid biopharmaceuticals, the amount of aggregation, Agg(T,t)=D(T,t). An example prediction for IL-1R(II) in a specified formulation are listed below,

| t = 2 years | 2° C. | 4° C. | 8° C. |
|---|---|---|---|
| Aggregation (%) | 3.4e⁻⁶ | 8.7e⁻⁶ | 5.9e⁻⁵ |

2. Given a specific level of unacceptable aggregation, the time it takes to reach that level. For example, if 1% aggregation will not be acceptable for IL-1R(II), the prediction for this time duration in the same formulation as in example 1, is greater than 10000 years. Note that in this example an acceptable shelf-life would have been attained (within a two-year duration). This happens to be an extraordinary optimal formulation for this protein therapeutic.

The new approach might also be used for other purposes. For example, it might be used to predict the effect of concentration on shelf life of a biologically active material. Or, it might be used to predict the effect of an excipient on shelf life of a biologically active material. To do this, the parameters could be determined for a composition comprising the biologically active material and one or more excipients. These parameters might then be extrapolated for use with the recommended storage temperature to yield the predicted shelf-life of the composition. These parameters might then be compared to predict the effect of the excipient on the shelf life of said biologically active material.

B. Apparatus

FIG. 9 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method of apparatus of the claims. Neither should the computing environment 100 be interpreted as having any dependency or requirement relating to any one or combination of components illustrated in the exemplary operating environment 100.

The steps of the claimed method and apparatus are operational with numerous other general purpose or special purpose computing system environments or configurations. Examples of well known computing systems, environments, and/or configurations that may be suitable for use with the methods or apparatus of the claims include, but are not limited to, personal computers, server computers, hand-held or laptop devices, multiprocessor Systems, microprocessor-based systems, set top boxes, programmable consumer electronics, network PCs, minicomputers, mainframe computers, distributed computing environments that include any of the above systems or devices, and the like.

The steps of the claimed method and apparatus may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In a distributed computing environment, program modules may be located in both local and remote computer storage media including memory storage devices.

With reference to FIG. 9, an exemplary system for implementing the steps of the claimed method and apparatus includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage, or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 9 illustrates operating system 134, application programs 135, other program modules 136, and program data 137.

The computer 110 may also include other removable/non-removable, volatile/nonvolatile computer storage media. By way of example only, FIG. 9 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 9 provide storage of computer readable instructions, data structures, program modules and other data for the computer 110. In FIG. 9, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball, or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and a printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 9. The logical connections depicted in FIG. 9 include a local area network (LAN) 171 and a wide area network (WAN) 173, but may also include other networks. Such networking environments are commonplace in offices, enterprise-wide computer networks, intranets, and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 9 illustrates remote application programs 185 as residing on memory device 181. It will be appreciated that the network connections shown are exemplary and other means of establishing a communications link between the computers may be used.

### CONCLUSIONS

The validity of the model in extracting meaningful thermodynamic parameters in a partially reversible system is determined by the capability of the model to determine these parameters uniquely. The level of sophistication in the model imposes limitations on the results. For example, if the model has too few parameters, one may either get a poor fit or forego certain detailed description of the system. On the other hand, if there are too many parameters, a good fit may not assure a unique set of parameters and therefore render them meaningless. Although global identifiability is difficult to achieve, we checked the local identifiability of our model by the rigorous calculation described above and showed that our calculated parameter values were all uniquely determined.

From the results we were able to extract meaningfully relevant thermodynamic (e.g. ΔC_{P}, ΔHₘand Tₘ) and kinetic parameters (e.g. A₁, E₁, A₂, E₂, A₃, E₃, A₄ and E₄) with varying levels of certainty. Two factors contribute to the uncertainty of the parameters we obtained. The first is the uncertainty in measurements. The second is the intrinsic sensitivity of the observables to each parameter under the conditions of the experiment. In order to estimate the uncertainties of the parameters in the model, we considered a 10% in protein concentration. Correspondingly the uncertainties induced by this error in the parameter estimations can be determined by projecting the error on the expectation space of theoretical observables. As a result of the quantity with the largest uncertainty is E₂, as shown in Table 1. This is expected since k₂ is least dependent on temperature amongst all the kinetic coefficients. Therefore in the limited temperature range of the experiment, we could not determine the value of E₂ and A₂ very well. However, despite this difficulty, we could still determine ΔHₘ and Tₘ relatively well. As suggested by this study, we expect improvement could be achieved if we expanded the study to include slower and faster DSC scan rates than those examined in the study. A more definitive improvement can be achieved if E₂ could be measured directly in a separate experiment.

We believe that the model presented, though not perfect, captures the main physical processes underlying the experimental conditions tested. Several points can now be made concerning the approximations and assumptions used as they pertain to real molecular properties. The model as presented tends to support the ΔHₘ=E₁-E₂ expectation. It can account for the additional 48 kcal/mol in the total enthalpy of the partially reversible system by allowing for populated D states (of aggregation) on the high temperature side of the unfolding envelope. This is supported by the massive aggregation observed at temperatures greater than or equal to the Tₘ. When k₃ and k₄ are set equal to zero to simulate the fully reversible case based on the theory, the Tₘ (˜53.5° C.) is very near that observed experimentally for the fully reversible case in urea (˜52.6° C.). The assumption that a combination of first and second order reaction rates are involved in the aggregation kinetics is supported by the experimental findings that the reaction order is ˜1.70±0.04. Finally, a reasonable prediction of aggregation rates at low temperatures (below the unfolding transition) was achieved taking into account curvature imposed by the ΔCₚ term used in the theoretical treatment. There is no question that further refinements based on more experimental evidence will help determine the mechanism and parameters more accurately so that extrapolation to other temperatures (above and below the Tₘ) result in meaningful predictions. The experimental findings suggest that stability of rhuIL1R (II) is afforded through the thermodynamic stabilization of the native state as suggested below the Tm, through the thermodynamic stabilization of the native state where progress to the irreversibly denatured aggregate is effectively blocked as in the case of the urea experiment. Furthermore, unfolded or conformationally altered protein propagate the aggregation reaction for this system.

It is anticipated that this model could be applied to better predict levels of aggregation at low temperatures. This aspect has significant implications with regard to fulfilling a need regarding better estimations of shelf-life for biopharmaceuticals. Furthermore, the model appropriately describes aggregation conditions associated with varying concentration factors. Hence, it is possible to run scan-rate dependent experiments at a single concentration in the calorimeter and translate the results into meaningful estimates that predict aggregation kinetics at other concentrations.

Having now fully described the invention, it will be appreciated by those skilled in the art that the invention can be performed within a range of equivalents and conditions without departing from the spirit and scope of the invention and without undue experimentation. In addition, while the invention has been described in light of certain embodiments and examples, the inventors believe that it is capable of further modifications. This application is intended to cover any variations, uses, or adaptations of the invention which follow the general principles set forth above.

The specification includes recitation to the literature and those literature references are herein specifically incorporated by reference.

The specification and examples are exemplary only with the particulars of the claimed invention set forth as follows:

1. A method for determining parameters for predicting aggregation kinetics of a biologically active material comprising the steps of:
(a) providing measurements of conformational change of the biologically active material at varying temperatures and varying times, and
(b) using the measurements of part (a) to mathematically determine activation energy parameters (E) and frequency factor parameters (A) associated with at least three different reaction rate constants, the parameters being predictive of aggregation kinetics of the biologically active material.

2. (canceled)

3. (canceled)

4. (canceled)

5. (canceled)

6. The method of claim 1 wherein one or more of the following equations is used:
\dot{N}=−k₁N+k₂U
\dot{U}=k₁N−(k₂+k₃)U−k₄U²
\dot{D}=k₃U+k₄U²

7. The method of claim 1 wherein the following equation is used:
$${C}_{P} ( v , T ) = ( Δ {H}_{m} + Δ {C}_{P} ( T - {T}_{m} ) ) ( - \frac{1}{v} \dot{N} ) + Δ {C}_{P} U + \frac{{k}_{3}}{v} ( {E}_{3} + Δ {C}_{P}^{{D}_{1}} ( T - {T}_{m} ) ) U + \frac{{k}_{4}}{V} ( {E}_{4} + {C}_{P}^{{D}_{2}} ( T - {T}_{m} ) ) {U}^{2} + ( Δ {C}_{P}^{{D}_{1}} + Δ {C}_{P} ) {D}_{1} + ( Δ {C}_{P}^{{D}_{2}} + Δ {C}_{P} ) {D}_{2}$$

8. (canceled)

9. (canceled)

10. (canceled)

11. The method of claim 1 wherein said determining step involves modeling aggregation, as a function of time at different temperatures, as a first and second order reaction.

12. The method of claim 1, in which at least some of the parameters collectively model non-Arrhenius aspects of the aggregation kinetics.

13. (canceled)

14. (canceled)

15. (canceled)

16. (canceled)

17. (canceled)

18. The method of claim 1 further comprising one or more steps of:
determining enthalpy or free energy of transition,
determining ΔCp, ΔC_{P}^{D}^{₁}, and ΔC_{P}^{D}^{₂}; or
determining the temperature at which about 50% of the protein is in an unfolded state and about 50% of the protein is in its native state.

19. (canceled)

20. The method of claim 1 wherein steps (a) and (b) are carried out on a plurality of different formulations of said biologically active material.

21. (canceled)

22. (canceled)

23. A method for predicting aggregation kinetics of a biologically active material comprising the steps of:
(a) providing activation energy parameter (E) and frequency factor parameters (A) associated with at least three different reaction rate constants, and
(b) predicting stability or aggregation kinetics as a function of time and temperature using at least three different reaction rate constants.

24. The method of claim 23 wherein the parameters of step (a) are determined by modeling aggregation, as a function of time at different temperatures, as a first and second order reaction, and
(i) using differential scanning calorimetry or size exclusion chromotography to provide measurements of conformational change of the biologically active material at varying temperatures and varying times;
(ii) providing estimated activation energy and frequency factor parameters;
(iii) calculating predicted measurements of conformational change based on the estimated parameters, and
(iv) using an estimation method to compare the predicted measurements to the measurements from step (i).

25. (canceled)

26. (canceled)

27. (canceled)

28. The method of claim 23 wherein said predicting step comprises predicting level of aggregation of said biologically active material at a temperature of 40 degrees C. or less.

29. The method of claim 28 wherein said temperature is in a range from 4 to 25 degrees C.

30. The method of claim 28 wherein said temperature is in a range from 15 to 30 degrees C.

31. The method of claim 28 wherein said temperature is in a range from −5 to 15 degrees C.

32. The method of claim 28 wherein said temperature is in a range from 2 to 8 degrees C.

33. The method of claim 23 wherein said predicting step comprises predicting level of aggregation of said biologically active material after a time period of three months or more.

34. The method of claim 33 wherein said time period is six months or more.

35. The method of claim 33 wherein said time period is nine months or more.

36. The method of claim 33 wherein said time period is one year or more.

37. The method of claim 33 wherein said time period is two years or more.

38. The method of claim 23 wherein the predicting step comprises predicting time to reach an unacceptable level of aggregation.

39. The method of claim 38 wherein the time to reach 50% aggregation is predicted.

40. (canceled)

41. (canceled)

42. (canceled)

43. The method of claim 23 wherein said predicting step comprises predicting stability or level of aggregation for a plurality of formulations of said biologically active material.

44. The method of claim 43 wherein at least one of the formulations contains one or more excipients.

45. The method of claim 43 wherein at least two of the formulations are at different pH.

46. The method of claim 23 wherein the effect of one or more excipients on shelf life of said biologically active material is predicted.

47. (canceled)

48. The method of claim 23 wherein said predicting step comprises using A_{gg}(T, t)=D.

49. (canceled)

50. (canceled)

51. (canceled)

52. A computer-readable medium having computer-executable instructions for determining parameters for predicting aggregation kinetics of a biologically active material, the instructions comprising the steps of:
(a) storing data of conformational change of the biologically active material at varying temperatures and varying times, and
(b) using the data of part (a) to mathematically determine activation energy parameters (E) and frequency factor parameters (A) associated with at least three different reaction rate constants, the parameters being predictive of aggregation kinetics of the biologically active material.

53. The computer-readable medium of claim 52 in which the instructions of step (b) comprises the steps of evaluating identifiability and variability of one or more of the parameters.

54. The computer-readable medium of claim 52 in which the instructions of step (b) comprise determining the change in heat capacity between native and denatured states of said biologically active material (ΔCp, ΔC_{P}^{D}^{₁}, ΔC_{P}^{D}^{₂}), wherein said change in heat capacity is predictive of aggregation kinetics of the biologically active material.

55. The computer-readable medium of claim 52 in which the instructions of step (b) comprise the steps of
(i) using estimated activation energy and frequency factor parameters,
(ii) calculating predicted measurements of conformational change based on the estimated parameters, and
(iii) using an estimation method to compare predicted measurements to the data from step (a).

56. The computer-readable medium of claim 55 in which the parameter estimation method is a non-linear least squares fitting method.

57. (canceled)

58. (canceled)

59. (canceled)

60. (canceled)

61. (canceled)

62. (canceled)

63. (canceled)

64. The computer-readable medium of claim 52 in which the data includes measurements of conformational change of the biologically active material under conditions that result in significant irreversible unfolding.

65. The computer-readable medium of claim 64 in which the data comprises data obtained from differential scanning calorimetry.

66. The computer-readable medium of claim 64 in which the data comprises data obtained from size exclusion chromatography.

67. The computer-readable medium of claim 52 in which the data comprises data of conformational change of the biologically active material measured as a function of temperature varied uniformly over time.

68. The computer-readable medium of claims 67 in which the instructions include applying a weighting factor dependent on the scan rate.

69. (canceled)

70. (canceled)

71. (canceled)

72. (canceled)

73. (canceled)

74. A computer readable medium having computer-executable instructions for predicting aggregation kinetics of a biologically active material comprising the steps of:
(a) storing activation energy parameter (E) and frequency factor parameters (A) associated with at least three different reaction rate constants, and
(b) predicting stability or aggregation kinetics as a function of time and temperature using at least three different reaction rate constants.

75. The computer readable medium of claim 74 in which the parameters of step (a) have been obtained by modeling aggregation, as a function of time at different temperatures, as a first and second order reaction, and
(i) using differential scanning calorimetry or size exclusion chromotography to provide measurements of conformational change of the biologically active material at varying temperatures and varying times;
(ii) providing estimated activation energy and frequency factor parameters;
(iii) calculating predicted measurements of conformational change based on the estimated parameters, and
(iv) using an estimation method to compare the predicted measurements to the measurements from step (i).

76. The computer readable medium of claim 74 wherein activation energy parameters (E) and frequency factor parameters (A) associated with at least four reaction rate constants are stored.

77. The computer-readable medium of claim 74 wherein activation energy parameters (E) and frequency factor parameters (A) associated with no more than four reaction rate constants are stored.

78. The computer-readable medium of claim 23 wherein said predicting step (b) comprises predicting level of aggregation of said biologically active material as a function of temperature, time and concentration of said biologically active material.

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. The computer-readable medium of claim 74 wherein said predicting step comprises predicting aggregation half-life of said biologically active material as a function of temperature and concentration of said biologically active material.

92. The computer-readable medium of claim 74 wherein said predicting step comprises predicting shelf-life of said biologically active material at one or more storage temperatures.

93. The computer-readable medium of claim 74 wherein said predicting step comprises predicting an optimal storage temperature.

94. (canceled)

95. (canceled)

96. (canceled)

97. (canceled)

98. The computer-readable medium of claim 74 further comprising instructions for selecting an optimal formulation.

99. (canceled)

100. The computer-readable medium of claim 52 wherein the data for the biologically active material is data for a protein.

101. The computer-readable medium of claim 100 wherein the data is for a hormone, cytokine, hematopoietic factor, growth factor, antibody, antiobesity factor, trophic factor, anti-inflammatory factor, antibody or enzyme.

102. The computer-readable medium of claim 101 wherein the data is for erythropoietin, granulocyte-colony stimulating factor, stem cell factor, or leptin.

103. A computing apparatus, comprising:
a display unit that is capable of generating video images;
an input device;
a processing apparatus operatively coupled to said display unit and said input device, said processing apparatus comprising a processor and a memory operatively coupled to said processor;
the processing apparatus being programmed to determine parameters for predicting aggregation kinetics of a biologically active material by performing steps comprising:
(a) storing measurements of conformational change of the biologically active material at varying temperatures and varying times, and
(b) using the measurements of part (a) to determine activation energy parameters (E) and frequency factor parameters (A) associated with at least three different reaction rate constants, the parameters being predictive of aggregation kinetics of the biologically active material.

104. The computing apparatus of claim 103, in which the processing apparatus is programmed with the steps of:
(i) receiving input of differential scanning calorimetry or size exclusion chromotography measurements of conformational change of the biologically active material at varying temperatures and varying times;
(ii) receiving input of estimated activation energy and frequency factor parameters;
(iii) calculating predicted measurements of conformational change based on the estimated parameters, and
(iv) using an estimation method to compare the predicted measurements to the measurements from step (i).

105. A computing apparatus, comprising:
a display unit that is capable of generating video images;
an input device;
a processing apparatus operatively coupled to said display unit and said input device, said processing apparatus comprising a processor and a memory operatively coupled to said processor;
the processing apparatus being programmed to predict aggregation kinetics of a biologically active material by performing steps comprising:
(a) storing activation energy parameter (E) and frequency factor parameters (A) associated with at least three different reaction rate constants, and
(b) predicting stability or aggregation kinetics as a function of time and temperature using at least three different reaction rate constants.

106. The computing apparatus of claim 105, in which processing apparatus is programmed with the steps of:
(i) receiving input of differential scanning calorimetry or size exclusion chromotography measurements of conformational change of the biologically active material at varying temperatures and varying times;
(ii) receiving input of estimated activation energy and frequency factor parameters;
(iii) calculating predicted measurements of conformational change based on the estimated parameters; and
(iv) using an estimation method to compare the predicted measurements to the measurements from step (i).
